(19) United States
(12) Patent Application Publication
(10) Pub. No.: US 2005/0038112 A1
(43) Pub. Date: Feb. 17, 2005

(54) Simplified sarcondictyn derivatives as anti-tumor agents

(76) Inventors: Nicola Mongelli, Tertulliano (IT); Maria Menichincheri, Casati (IT); Marina Ciomei, Lorenteggio (IT); Cesare Gennari, Lamarmora (IT); Joachim Telser, Wuppertal (DE); Raphael Beumer, Neudorfer Str (DE)

Correspondence Address: Lisa M W Hillman;McDonnell Boehnen Hulbert & Berghoff, 300 South Wacker Drive, Chicago, IL, 60606, US

(21) Appl. No.: 10/489,508

(22) PCT Filed: Sep. 6, 2002
(86) PCT No.: PCT/EP02/10038

(30) Foreign Application Priority Data
Sep. 14, 2001 (GB) 0122257.9

(51) Int. Cl. C07C 69/74; C07C 49/293; A61K 31/215; A61K 31/12

(52) U.S. Cl. 514/529; 514/691; 560/119; 568/374

(57) ABSTRACT

There are provided sarcodictyin derivatives or pharmaceutically acceptable salts thereof, which are characterized by a simplified chemical structure and have anti-tumor activity. A process for their preparation, the pharmaceutical compositions containing them, and their use in the prevention, control and treatment of cancer are also provided.

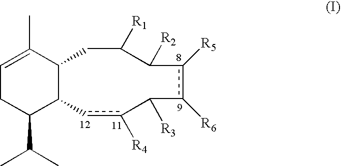

The present invention relates to new anti-tumor agents, to a process for their preparation, to pharmaceutical compositions containing them, and to the use of such compounds in the prevention, control and treatment of cancer. Sarcodictyins A and B were isolated in 1987 by Pietra et al. from the Mediterranean stoloniferan coral Sarcodictyon roseum, while their anti-tumor activity was recognised about a decade later, and their paclitaxel-like mechanism of action uncovered (Ciomei, M. et al. Proc. Amer. Ass. Canc. Res. 1997, 38, 5 and WO 96 36 335-A1)

In the meantime, the diterpene glycoside eleutherobin was reported by Fenical et al. from an Eleutherobia species of australian soft coral, accompanied by disclosure of its potent cytotoxicity. Two years later, in 1997, it was shown that eleutherobin, similarly to sarcodictyins, acted by mitotic arrest through induced tubulin polymerization.

Now, there is a strong need for more simplified molecules, which nevertheless maintain the useful properties referred to above characterizing the natural substances.

The present invention relates to a new class of simplified sarcodictyins. In particular, the present invention provides a compound which is a sarcodictyin derivative of formula (I) 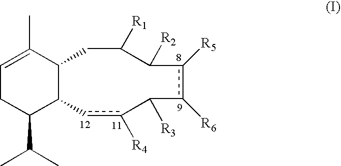
wherein: 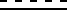
at positions 8-9 and 11-12 independently represents a single or double bond, —R₁ represents oxygen (═O), or a residue —OR₇, wherein R₇ represents hydrogen, linear or branched C₁-C₇ alkanoyl, benzoyl, C₁-C₁₀ alkyl, C₂-C₁₀ alkenyl or a residue of the formula 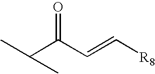
wherein R₈ is an optionally substituted aryl or heterocyclyl; one of —R₂ and —R₃ represents hydrogen and the other one is chosen from the group consisting of hydrogen, oxygen (═O) and a residue —OR₉, wherein R₉ represents hydrogen, C₁-C₆ alkanoyl or benzoyl;
when 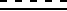
at position 11-12 represents a single bond, then —R₄ represents oxygen (═O), methylene (═CH₂), ═CHCOOR₁₀, wherein R₁₀ represents C₁-C₁₀ alkyl or optionally substituted aryl; ═CH(OCH₃), or a residue of formula
—OR₉, wherein R₉ is as defined above;
—CH₂OR₁₁ wherein R₁₁ represents hydrogen or a sugar residue,
—COR₁₂ wherein R₁₂ represents hydrogen, —OH or —OR₁₀, wherein R₁₀ is as defined above; or
when 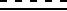
at position 11-12 represents a double bond, then —R₄ represents a residue of formula —CH₂OR₁₁ or —COR₁₂ as defined above;
—R₅ and —R₆ are both hydrogen or, when 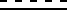
at position 8-9 represents a single bond, taken together with the carbon atoms to which they are attached form a cyclopropane ring; or a pharmaceutically acceptable salt thereof.

As used herein the term “C₁-C₁₀ alkyl” refers to a straight or branched chain alkyl moiety having from 1 to 10 carbon atoms, including for example, methyl, ethyl, propyl, isopropyl, n-butyl, i-butyl, sec-butyl, tert-butyl, n-pentyl, isopentyl, n-hexyl, n-heptyl and n-octyl. The term “C₂-C₁₀ alkenyl” as used herein refers to a straight or branched chain alkenyl moiety having from 2 to 10 carbon atoms and having in addition one double bond of either E or Z stereochemistry where applicable. Examples of alkenyl groups include: vinyl, allyl, metallyl, butenyl and crotyl. The term “aryl” as used herein refers to a monocyclic or bicyclic aromatic hydrocarbon group of 6 to 10 carbon atoms, such as phenyl, naphthyl, indanyl; furthermore, “aryl” as used herein may refer to a diphenyl group (—C₆H₄—C₆H₅). The term “C₁-C₇ alkanoyl” refers to acyl residues such as formyl, acetyl, pivaloyl (PIV), and pentanoyl groups.

The term “heterocyclyl” as used herein refers to a 3- to 7-membered, substituted or unsubstituted, saturated or unsaturated heterocyclyl ring, containing at least one heteroatom selected from O, S and N, any ring carbon may be oxidized as a carbonyl, and wherein said heterocyclyl ring may be optionally fused to a second 5- or 6-membered, saturated or unsaturated heterocyclyl ring, or to a C₃-C₇ cycloalkyl ring, or to a benzene or naphthalene ring. Examples of heterocyclyl groups are pyrrolyl, pyrrolidinyl, pyrazolyl, imidazolyl, triazolyl, tetrazolyl, oxazolyl, isoxazolyl, thiazolyl, isothiazolyl, thiadiazolyl, thienyl, tetrahydrothienyl, furyl, tetrahydrofuryl, aziridinyl, oxiranyl, azetidinyl, succinimido, pyridyl, piperidinyl, pyrazinyl, piperazinyl, pyridazinyl, hexahydropyridazinyl, pyrimidinyl, pyranyl, tetrahydropyranyl, benzothienyl, benzothiazolyl, benzoxazolyl, isobenzofuranyl, benzofuranyl, benzimidazolyl, indazolyl, chromenyl, indolyl, oxindolyl, phthalimido, 1-oxo-2-isoindolyl, quinolyl, isoquinolyl, tetrahydroisoquinolyl, indolizinyl, isoindolyl, 2-oxoisoindolyl, 1,2-(methylenedioxy)phenyl, quinuclidinyl, hydantoinyl, saccarinyl, cinnolinyl, purinyl, morpholinyl, thiomorpholinyl, dioxanyl, dithianyl and azepinyl. Most preferred heterocyclyl groups are N-methyl-imidazolyl, 2-methyl-thiazolyl, 2-methyl-oxazolyl and pyridyl group. Preferably, when OR₁₁ is a sugar residue, it has the formula 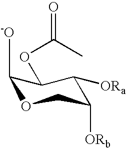
wherein Rₐ and R_{b} independently represent hydrogen, a hydroxy protecting group, or C₁-C₆ alkanoyl.

Substituents which may be present in the aryl or heterocyclyl groups in any of the above definitions of R₁-R₁₀ include the following:
halo (i.e., fluoro, bromo, chloro or iodo);
hydroxy;
nitro;
azido;
mercapto (i.e., —SH), and acetyl or phenylacetyl esters thereof (i.e., —SCOCH₃ and —SCOCH₂C₆H₅);
amino (i.e., —NH₂ or —NHR^{I} or —NR^{I}R^{II}, wherein R^{I} and R^{II}, which are the same or different, are straight or branched C₁-C₆ alkyl, phenyl, biphenyl (i.e., —C₆H₄—C₆H₅), or benzyl groups, optionally substituted by hydroxy, methoxy, methyl, amino, methylamino, dimethylamino, chloro or fluoro; or R^{I} and R^{II} taken together with the nitrogen atom to which they are attached form a heterocyclic ring such as morpholino, pyrrolidino, piperidino, pyperazino or N-methylpyperazino;
guanidino, i.e., —NHC(═NH)NH₂;
formyl (i.e. —CHO);
cyano;
carboxy (i.e. —COOH), or esters thereof (i.e., —COOR^{I}), or amides thereof (i.e., —CONH₂, —CONHR^{I} or —CONHR^{I}R^{II}), wherein R^{I} and R^{II} are as defined above, and including morpholino-amides, pyrrolidino-amides, and carboxymethylamides —CONHCH₂COOH;
sulfo (i.e., —SO₃H);
acyl, i.e., —C(O)R^{I}, wherein R^{I} is as defined above, including monofluoroacetyl, difluoroacetyl, trifluoroacetyl;
carbamoyloxy (i.e., —OCONH₂) and N-methylcarbamoyloxy,
acyloxy, i.e., —OC(O)R^{I} wherein R^{I} is as defined above, or formyloxy,
acylamino, i.e., —NHC(O)R^{I}, or —NHC(O)OR^{I}, wherein R^{I} is as defined above or is a group CH₂)ₜCOOH where t is 1, 2 or 3;
ureido, i.e., —NH(CO)NH₂, —NH(CO)NHR^{I}, —NH(CO)NR^{I}R^{II}, wherein R^{I} and R^{II} are as defined above, including —NH(CO)-(4morpholino), —NH(CO)-(1-pyrrolidino), —NH(CO)(1-piperazino), —NH(CO)-(4-methyl-1-piperazino);
sulfonamido, i.e., —NHSO₂R^{I} wherein R^{II} is as defined above;
   a group —(CH₂)ₜCOOH, and esters and amides thereof, i.e., —(CH₂)ₜCOOR^{I} and —(CH₂)ₜCONH₂, —(CH₂)ₜCONHR^{I}, —(CH₂)ₜCONR^{I}R^{II}, wherein t, R^{I} and R^{II} are as defined above;
   a group —NH(SO₂)NH₂, —NH(SO₂)NHR^{I}, —NH(SO₂)NR^{I}R^{II}, wherein R^{I} and R^{II} are as defined above, including —NH(SO₂)-(4-morpholino), —NH(SO₂)-(1-pyrrolidino), —NH(SO₂)-(1-piperazino), —NH(SO₂)-(4-methyl-1-piperazino);
   a group —OC(O)OR^{I}, wherein R^{I} is as defined above;
   a group —OR^{I}, wherein R^{I} is as defined above, including —OCH₂COOH;
   a group —SR^{I}, wherein R^{I} is as defined above, including —SCH₂COOH;
   a group —S(O)R^{I}, wherein R^{I} is as defined above;
   a group —S(O₂)R¹, wherein R^{I} is as defined above;
   a group —SO₂NH₂, —SO₂NHR^{I}, or —SO₂NR^{I}R^{II}, wherein R^{I} and R^{II} are as defined above;
   C₁-C₆ allyl or C₂-C₆ alkenyl;
   C₃-C₇ cycloalkyl;
   substituted methyl selected from chloromethyl, fluoromethyl, difluoromethyl, trifluoromethyl, aminomethyl, N,N-dimethylaminomethyl, azidomethyl, cyanomethyl, carboxymethyl, sulfomethyl, carbamoylmethyl, carbamoyloxymethyl, hydroxymethyl, methoxycarbonylmethyl, ethoxycarbonylmethyl, tert-butoxycarbonylmethyl and guanidinomethyl.

When present, carboxy, hydroxy, mercapto and amino groups may be either free or in a protected form. Protected forms of said groups are any of those generally known in the art. Preferably, carboxy groups are protected as esters thereof, in particular methyl, ethyl, tert-butyl, benzyl, and 4-nitrobenzyl esters. Preferably, hydroxy groups are protected as silyl-ethers, ethers or esters thereof, in particular trimethyl silyl, tert-butyldiphenyl silyl, triethyl silyl, triisopropyl silyl or tert-butyldinethylsilyl ethers, methoxymethyl ethers, tetrahydropyranyl ethers, benzyl ethers, acetates or benzoates. Preferably, mercapto groups are protected as thioethers or thioesters, in particular tert-butyl thioethers, thioacetates or thiobenzoates. Preferably, amino groups are protected as carbamates, e.g. tert-butoxycarbonyl derivatives, or as amides, e.g. acetamides and benzamides.

As stated above, the present invention provides the salts of those compounds of formula (I) that have salt-forming groups, especially the salts of the compounds having a carboxylic group or the salts of the compounds having a basic group, especially an amino. The salts are especially physiologically tolerable salts, for example alkali metal and alkaline earth metal salts (e.g. sodium, potassium, lithium, calcium and magnesium salts), ammonium salts and salts with an appropriate organic amine or amino acid (e.g. arginine, procaine salts), and the addition salts formed with suitable inorganic acids (e.g. hydrochlorides, hydrobromides, sulfates, phosphates) or carboxylic and sulfonic organic acids (e.g. acetates, trifluoroacetates, citrates, succinates, malonates, lactates, tartrates, fumarates, maleates, methanesulfonates, p-toluenesulfonates).

Furthermore, hydrates, solvates of compounds of formula (I), and physiologically hydrolyzable derivatives (i.e., prodrugs) of compounds of formula (I) are included within the scope of the present invention.

It is to be noted that the R₁, R₂, R₃ and R₄ substituents may be above or under the plane, so that the present invention encompasses all the possible stereoisomers (e.g. diastereoisomers, epimers, geometrical isomers) of the compounds of formula (I), as well as their racemic or optically active mixtures related to these substituents.

In the preferred configuration, the substituent R₁ is under the plane:

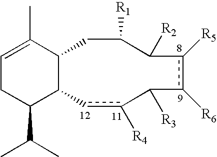

A preferred sarcodictyin derivative of the present invention has the following formula (Ia) 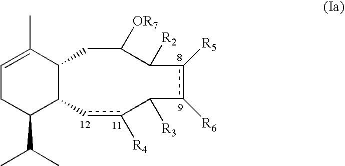
wherein: 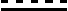
at positions 8-9 and 11-12 independently represents a single or double bond, R₇ represents a residue of the formula 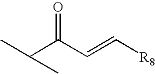
wherein R₈ is N-methyl imidazolyl, phenyl, methyl-thiazolyl, methyl-oxazolyl or pyridyl group;
one of —R₂ and —R₃ represents hydrogen and the other one is hydrogen or oxygen (═O), hydroxy, acetoxy;
when 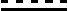
at position 11-12 represents a single bond, then —R₄ represents oxygen (═O), methylene (═CH₂), ═CHCOOR₁₀, wherein R₁₀ represents methyl or ethyl, ═CH(OCH₃), —CHO; hydroxy, acetoxy, pivaloyloxy group or —CH₂OR₁₁ wherein R₁₁ represents hydrogen or a sugar residue having the formula 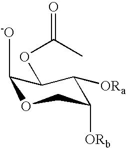
wherein Rₐ and R_{b} independently represent hydrogen, a hydroxy protecting group, or C₁-C₆ alkanoyl, or
when 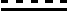
at position 11-12 represents a double bond, then —R₄ represents a residue of formula —CO₂C₂H₅; and
—R₅ and —R₆ are both hydrogen atoms or, when 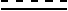
   at position 8-9 represents a single bond, taken together with the carbon atoms to which they are attached form a cyclopropane ring. The present invention also provides a process for preparing a compound of the invention as defined above, which process comprises:
cyclizing a compound of formula II 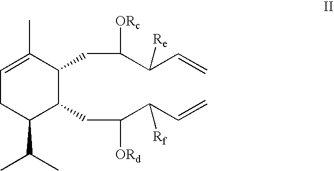
wherein R_{c} represents hydrogen, a silyl protecting group, C₁-C₆ alkanoyl or benzoyl or, taken together with Rₑ, forms an acetonide ring; R_{d} represents hydrogen, C₁-C₆ alkanoyl, or benzoyl, or, taken together with R_{f}, forms an acetonide ring; either Rₑ represents H and R_{f} either represents OH or is linked to the adjacent OR_{d} substituent as defined above, or R_{f} represents H and Rₑ either represents OH or is linked to the adjacent OR_{c} substituent as defined above;
and, if desired, converting the resulting sarcodictyin derivative of formula I′, 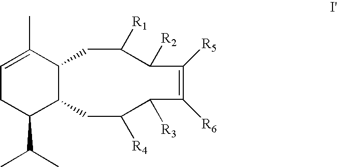
wherein R₁ is OR_{c}, R₂ is Rₑ, R₃ is R_{f}, R₄ is OR_{d}, in which R_{c}, R_{d}, Rₑ and R_{f} are as defined above and R₅ and R₆ are hydrogen atoms, into another sarcodicytin derivative of formula I as defined above; and/or if desired, converting a sarcodictyin derivative of formula I′ or I into a pharmaceutically acceptable salt therof; and/or, if desired converting a pharmaceutically acceptable salt of a sarcodictyin derivative of formula I or I′ into the corresponding free compound.

Preferably, R_{c} represents a silyl protecting group, more preferably a t-butyldiphenylsilyl group. The cyclization to give the compound of formula I′ as single Z isomer can be performed through the Ring Closing Metathesis (RCM) reaction. In particular, the RCM reaction is carried out in the presence of an appropriate catalyst, more preferably a Nolan and Grubb's catalyst, described for example in J.Am.Chem.Soc., 1999, 121, 2674 and in Org. Lett., 1999, 1, 953. The conversion of a compound of formula I′ or I into another different final compound of formula I may be carried out in several ways, depending on the meanings of the substituents and the presence of the unsaturated bonds in the ring. Such conversions follow conventional procedures which are known in the art.

For example, a compound of formula I wherein —R₁ represents a residue of the formula 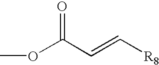
wherein R₈ is as defined above, can be obtained by condensing a corresponding compound of the formula I or I′ wherein —R₁ represents hydroxy group with an activated form of the appropriate derivative of formula 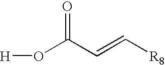
wherein R is as above defined.

A compound of formula I wherein —R₂, —R₃ or —R₄ represents an oxygen atom ═O can be obtained from a corresponding compound of formula I or I′ as defined above wherein —R₂, —R₃ or —R₄ represents a hydroxy group by means of oxidation, for example with Dess-Martin periodinane, PDC or PCC or under Swern oxidation conditions (DMSO/oxalyl chloride), provided that the other hydroxy groups in the molecule, if any, are protected. A compound of formula I wherein —R₄ represents an oxygen atom ═O can be conveniently converted into a corresponding compound of formula I wherein —R₄ represents methylene (═CH₂), ═CHCOOR₁₀ wherein R₁₀ is as defined above, or ═CH(OCH₃) by reaction with a suitable Wittig reagent, such as for example, respectively, Ph₃P═CH₂, Ph₃P═CHCOOR₁₀, wherein R₁₀ is as defined above and Ph₃P═CH(OCH₃). A compound of formula I wherein —R₄ represents ═CH(OCH₃) can be then converted by acidic hydrolysis into a corresponding compound of formula I wherein —R₄ represents —CHO, which in turn may be either reacted with a reducing agent to give a compound of formula I wherein —R₄ represents —CH₂OH, or oxidised with a suitable reagent such as NaClO₂ to give a compound of formula I wherein —R₄ represents —COOH. A compound of formula I wherein —R₄ represents an oxygen atom ═O can also be converted into a compound of formula I wherein —R₄ represents a —COOR₁₀ group wherein R₁₀ is as defined above and the bond at position 11-12 is double by treatment with triflic anhydride in the presence of a base followed by reaction of the resultant enol-triflate with CO and R₁₀—OH wherein R₁₀ is as defined above in the presence of Palladium catalyst and a base such as triethylamine according to known procedures as those described in J.Chem.Soc.Perkin Trans. I, 1991 (5), 969-979. Such compounds of formula I wherein —R₄ represents a —COOH group and the bond at position 11-12 is double can be converted by selective reduction into the corresponding 11-12 unsaturated compounds of formula I wherein —R₄ represents a —CH₂OH group, for example by treatment with ClCOOEt/NaBH₄.

A compound of formula I′ or I wherein the bond at position 8-9 is double may be converted into the corresponding compound of formula I with a single bond at the 8-9 position and wherein R₅ and R₆ are hydrogen atoms by hydrogenation, such as by treatment with H₂/(Ph₃P)₃RhCl (Ireland, R. E.; Bey, P. Org. Synth. 1973, 53, 63; Osborn, J. A.; Wilkinson, G. Inorg. Synth. 1977, 28, 77) or with diimide (NH═NH, Org.React. 1991, 40, 91); or into the corresponding compounds of formula I with a single bond at the 8-9 position wherein R₅ and R₆ taken together with the carbon atoms to which they are attached, form a cyclopropane ring by treatment with a suitable reactant such as a zinc carbenoid (J.Am.Chem.Soc. 2001, 123, 8139-8140).

A sarcodictyin derivative may be converting into a pharmaceutically acceptable salt thereof by salification, using conventional techniques. Suitable salts include those mentioned above. A compound of the formula II may be prepared as described in any one of the following scheme, in which R_{c} and R_{d} have the meanings above defined:

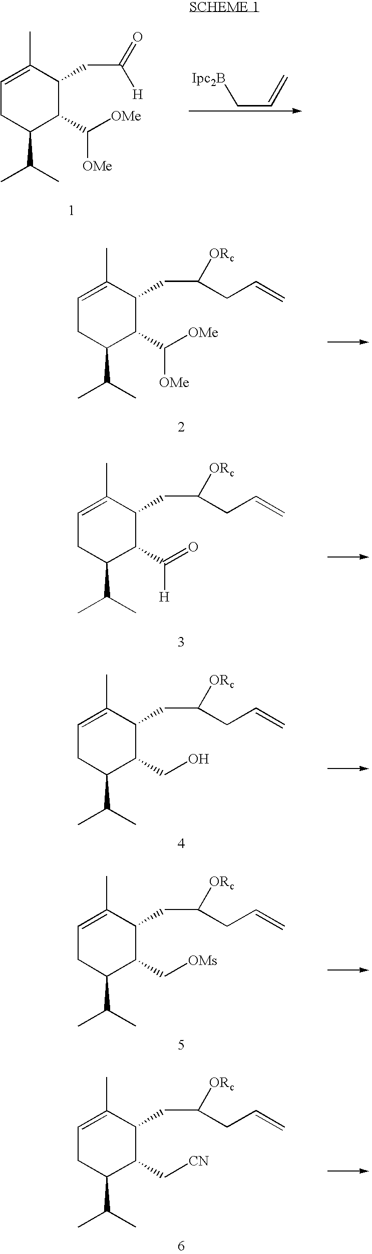

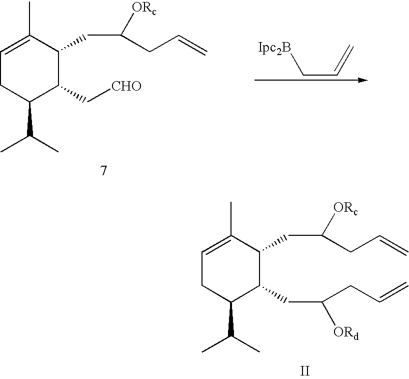

The starting compound 1 was submitted to Brown's stereoselective allylation reaction, (J. Org. Chem. 1982, 47, 5065) generating the oxygenated stereocenter of the compound 2. The allylation reaction proceeds with good to excellent stereocontrol in favor of the desired stereoisomer, just by choosing the suitable absolute stereochemistry of the alpha-pinene-derived reagent [(1-Ipc from (−)-alpha-pinene or d-Ipc from (+)-alpha-pinene]. After standard alcohol protection, such as t-butyldiphenylsilylation, an efficient and well established sequence of steps—dimethylacetal hydrolysis; aldehyde reduction; alcohol mesylation; mesylate substitution with cyanide; nitrile reduction to aldehyde-led to the homologated aldehyde 7, on which the same allylation procedure described above was applied. The allylation reaction proceeds on aldehyde 7 with good to excellent stereocontrol in favor of the desired stereoisomer, just by choosing the suitable absolute stereochemistry of the alpha-pinene-derived reagent [(1-Ipc from (−)-alpha-pinene or d-Ipc from (+)-alpha-pinene]. The starting compounds of formula I are known or can be obtained from known compounds in a manner analogous to that described for example in Tetrahedron Lett. 1999, 40, 153.

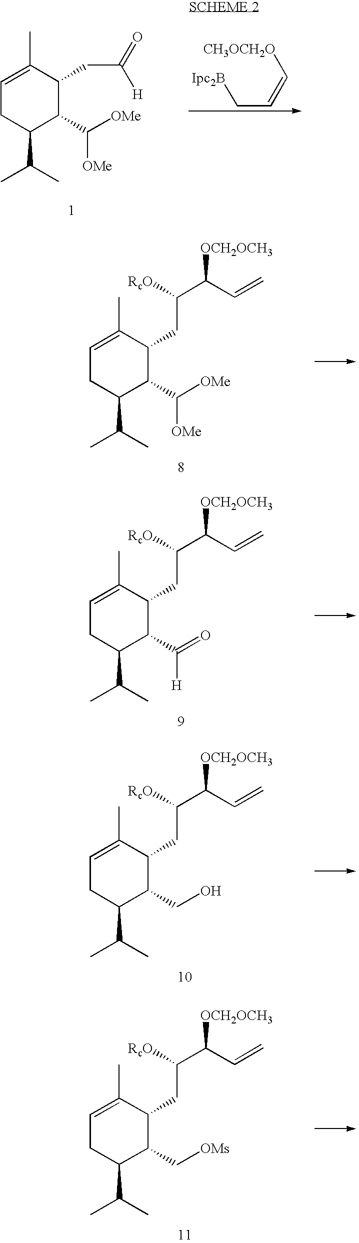

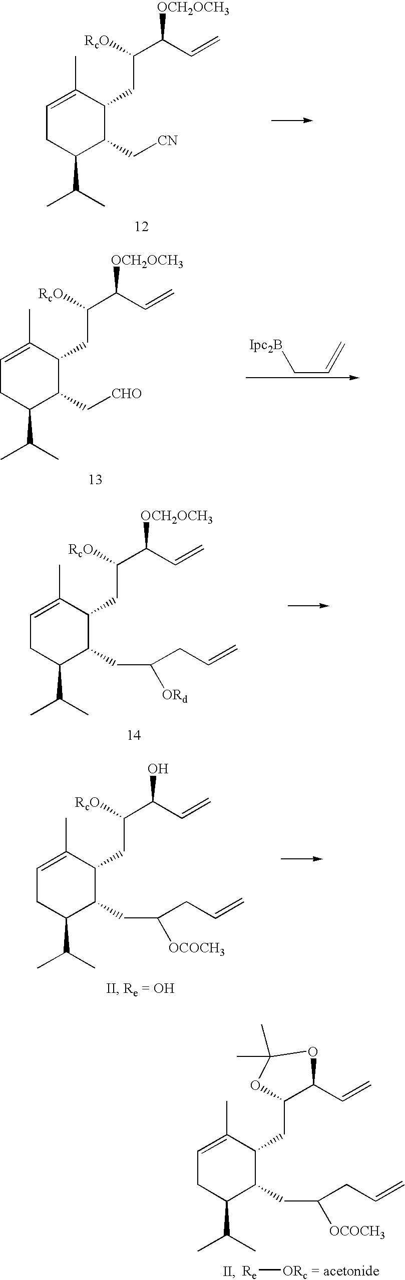

Addition of the oxyallyl borane [(1-Ipc from (−)-alpha-pinene] to aldehyde 1 gave the alcohol 8 (Rc=H) in good to excellent stereocontrol. Choosing the opposite absolute stereochemistry of the alpha-pinene-derived reagent [d-Ipc from (+)-alpha-pinene] gives access to the opposite diastereoisomer (OR_{c} up, OCH₂OCH₃ down).

Protection of the secondary alcohol as a tert-butyldiphenylsilylether, followed by the reaction sequence already mentioned above for the transformation of 2 to 7 (deprotection of the acetal, reduction of the liberated aldehyde and C₁-chain elongation via substitution of the mesylate by cyanide) gave after reduction the aldehyde 13. Addition of the desired allyl borane gave the secondary alcohol (14, R_{d}═OH), which was protected with acetic anhydride, yielding the acetate (14, R_{d}═COCH₃). Deprotection of the MOM-group (CH₃OCH₂—) leads to free allylic alcohol II (Rₑ═OH). As stated above, also in this last allylation reaction proceeds with good to excellent stereocontrol in favor of the desired stereoisomer, just by choosing the suitable absolute stereochemistry of the alpha-pinene-derived reagent [(1-Ipc from (−)-alpha-pinene or d-Ipc from (+)-alpha-pinene].

In alternative, an acetonide group is introduced to constrain one side chain. Deprotection of the intermediate and subsequent protection of the resultant diol (II, R_{c}═H, Rₑ═OH) gives the acetonide of formula II, wherein Rₑ and the OR_{c} group taken together form an acetonide ring.

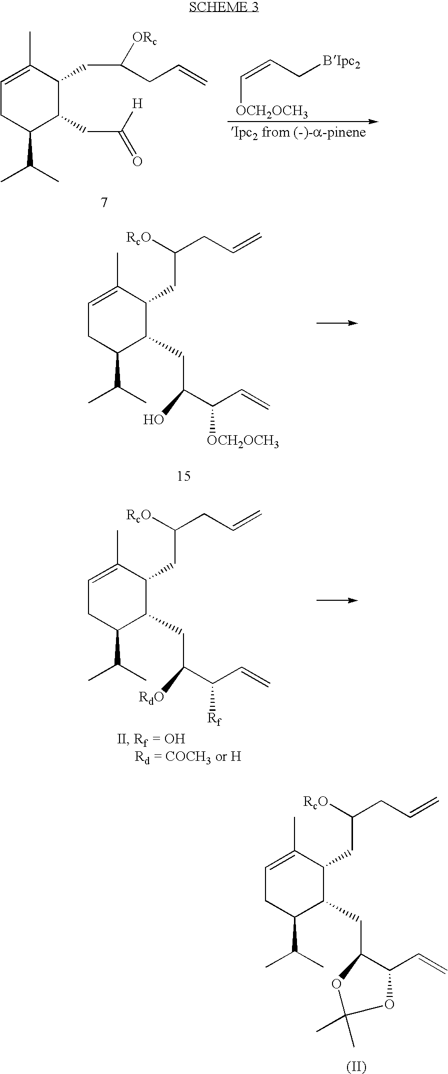

The compounds formula II wherein R_{f} is hydroxy group are prepared starting from the aldehyde 7 with the desired configuration through reactions analogous to those mentioned above. The compounds of formula II can be converted into the corresponding diol derivative by deprotection (II, R_{d}═H, R_{f}═ROH), which can be further transformed into the acetonide derivative (II, R_{f}—OR_{d}=acetonide).

Biological Tests

Cytotoxicity

A2780 cells (2000/well) were seeded in multiwell plates (96 wells) in the presence of 200 μl of the complete medium RPMI 1640+10% FCS. After 24 h, the cells were treated with the compounds: the compounds' solution (200×) was prepared in DMSO 100% and 1 μl/well was added. 5 scalar concentrations for each compound were tested in four replicates. The cells were incubated at 37° C., 5% CO₂ for 72 h.

Colorimetric assay (SRB: sulforhodamine B): cell cultures were fixed with trichloroacetic acid, stained with 0.4% SRB dissolved in 1% acetic acid. Unbound dye was removed by four washes with 1% acetic acid and protein-bound dye was extracted with 10 mM Tris base for determination of optical density in a 96-well microtiter plate reader. IC₅₀ and IC₉₀ (concentration inhibiting cell proliferation by 50 or 90%) were determined by data analysis in the Microsoft Excel 97 program.

Effect on Cell Cycle Progression

Human colon carcinoma HCT116 cells were seeded in culture flasks and treated 24 h after incubation at 37° C. At the end of the treatment (24 or 48 or 72 hours), cells were counted and resuspended in propidium iodide (PI) staining solution (0.1% sodium citrate, 0.1% nonidet P40, 6.5 μg/ml Rnasi A, 50 μg/ml PI). After incubation in the dark at room temperature for at least 30 minutes, samples were then analyzed for cell cycle on FacScan (Becton Dickinson) flow cytometer.

Human colon carcinoma HT29 cells were also used to study the cytotoxicity of the compounds of the present invention.

TABLE 1
Cytotoxicity tests: IC₅₀ values on several different
tumor cell lines (A2780; HCT116; HT29)

| Compound | | | |
|---|---|---|---|
| prepared in | IC₅₀ [μM] | IC₅₀ [μM] | IC₅₀ [μM] |
| Example | (A2780) | (HCT116) | (HT29) |
| 12 | 4 | 12 | — |
| 16 | 40 | 35-60 | 30 |
| 19 | 4 | 5 | 6 |
| 27 | <5 | 10-25 | 7 |
| 34 | 4 | 4 | 5 |

Microtubule Assembly and Disassembly Assay.

Pig brain tubulin was prepared by two cycles of assembly and disassembly and it was stored in liquid nitrogen in Microtubule Assembly Buffer (MAB: 0.1 M MES, 2.5 mM EGTA, 0.5 mM MgSO₄, 0.1 mM EDTA, 0.1 mM DTT pH 6.4). Assembly was monitored by the method of Gaskin et al. (Gaskin F, Cantor C R, Shelanski M L, 1974,: Turbidimetric studies of the in vitro assembly and disassembly of porcine neurotubules. J. Mol. Biol. 89: 737-758). The cuvette (1 cm path) containing 0.5 mg/ml tubulin and 1 mM GTP was shifted to 37° C. and continuous turbidity measurements were made at 340 nm on a spectrophotometer equipped with an automatic recorder and a thermostatically regulated sample chamber. After 30 min CaCl₂ (5 mM) was added and disassembly was monitored for 10 mm as decreased turbidity. Scalar doses of test compounds were monitored at regular intervals of 15 min.

Data were expressed as percentage of reassembly induced by the tested compounds and the dose effecting tubulin assembly by 50% or 90% at 37° C. (ED₅₀ and ED₉₀) was calculated on this curve.

TABLE 2
Tubulin polymerizing activities: ED₅₀ = effective dose that
induces 50% tubulin polymerisation; ED₉₀ = effective dose
that induces 90% tubulin polymerisation

| Compound | | |
|---|---|---|
| prepared in | | |
| Example | ED₅₀ [μM] | ED₉₀ [μM] |
| 12 | 2.0 | 10.0 |
| 16 | 0.2 | 1.2 |
| 19 | 5.0 | 16.0 |
| 27 | 3.0 | 7.0 |
| 30 | 1.0 | 1.7 |
| 34 | 1.0 | 1.8 |

Compounds of formula I of the invention show enhanced antitumor activity and acceptable toxicity. They are useful as antitumour agents in the prevention, treatment and/or control of cancer, for instance in the treatment of leukemia and solid tumors, such as colon, colorectal, ovarian, mammary, prostate, lung, kidney and also melanoma tumors. A human can be treated by a method comprising administering thereto a therapeutically effective amount of a compound of the invention. The invention therefore provides a method of treating a patient in need of an antitumour agent, which method comprises the administration thereto of a compound as defined above. The condition of the human patient can thus be improved. The invention also provides the use of a compound of the invention as defined above in the manufacture of a medicament for use as an antitumour agent.

The dosage range adopted will depend on the route of administration and on the age, weight and condition of the patient being treated. The compound of formula (I) is typically administered by parenteral route, for example intramuscularly, intravenously or by bolus infusion. A suitable dose range is from 1 to 1000 mg of equivalent per m² body surface area of active drug, for instance from 10 to 500 mg/m².

The compounds of formula (I) may be formulated into a pharmaceutical composition together with a pharmaceutically carrier or diluent. The invention therefore further provides a pharmaceutical composition which comprises a pharmaceutically acceptable diluent or carrier and, as an active ingredient, a compound as defined above. The pharmaceutical compositions of the invention are prepared by conventional methods and are adminstered in a pharmaceutically acceptable form. For example, the solid oral forms may contain, together with the active compound, diluents, e.g. lactose, dextrose, saccharose, sucrose, cellulose, corn starch or potato starch; lubricants, e.g. silica, talc, stearic, magnesium or calcium stearate, and/or polyethylene glycols; binding agents, e.g. starches, arabic gum, gelatine, methylcellulose, carboxymethylcellulose or polyvinyl pyrrolidone; disaggregating agents, e.g. a starch, alginic, alginates or sodium starch glycolate; effervescing mixtures; dyestuffs; sweeteners; wetting agents such as lecithin, polysorbates, laurylsulphates; and, in general, non-toxic and pharmacologically inactive substances used in pharmaceutical formulations. Said pharmaceutical preparations may be manufactured in known manner, for example, by means of mixing, granulating, tabletting, sugar-coating, or film-coating processes.

The liquid dispersions for oral administration may be e.g. syrups, emulsions and suspensions.

The syrups may contain as carrier, for example, saccharose or saccharose with glycerine and/or mannitol and/or sorbitol.

The suspensions and the emulsions may contain as carrier, for example, a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusions may contain as carrier, for example, sterile water or preferably they may be in the form of sterile, aqueous, isotonic saline solutions or they may contain as a carrier propylene glycol.

The suppositories may contain together with the active compound a pharmaceutically acceptable carrier, e.g. cocoa butter, polyethylene glycol, a polyoxyethylene sorbitan fatty ester surfactant or lecithin.

Typically the pharmaceutical compositions are formulated for parenteral administration, for example by dissolution in water for injection or physiological saline.

### EXAMPLE 1

(R)-1-((1R,5R,6R)-6-Dimethoxymethyl-5-isopropyl-2-methyl-cyclohex-2-enyl)-pent-4-en-2-ol.

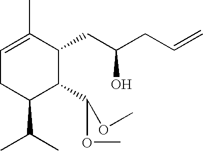

Allyl magnesium bromide (1M in Et₂O, 610 μl, 0.61 mmol, 1.2 eq) was added slowly to a stirred 0.86 M solution of ¹Ipc₂BOMe (¹Ipc derived from (−)-alpha-pinene) in THF (830 μl, 0.71 mmol, 1.4 eq) at 0° C. in an oven dried Schlenck tube under an argon atmosphere. The mixture was stirred at ambient temperature for 1 h, then cooled to −78° C. and a solution of 149 mg (0.51 mmol) of [(1R,5R,6R)-6-dimethoxymethyl-5-isopropyl-2-methyl-cyclohex-2-enyl]-acetaldehyde, prepared as described in Ceccarelli S. et al, Tetrahedron Lett. 1999, 40, 153, dissolved in 2 ml of anhydrous ether, was added dropwise via syringe. The reaction mixture was stirred at −78° C. for 6 h, then warmed to room temperature and quenched with 250 μl of 6M NaOH and 200 μl of 35% H₂O₂. The mixture became hot spontaneously and was left standing over night. The title product was isolated and purified by intensive flash chromatography (Hex/EtOAc 4+1), R_{f}=0.43. Yield: 117 mg (0.39 mmol, 77%) as colourless oil.

R_{f}=0.43 (hexane/EtOAc 4:1); ¹H NMR (200 MHz, CDCl₃): δ=5.98-5.77 (m, 1H), 5.36 (m, 1H), 5.16-5.12 (m, 1H), 5.07 (br s, 1H), 4.36 (d, J=5.4 Hz, 1H), 3.95-3.86 (m, 1H), 3.38 (s, 6H), 2.46-1.19 (m, 14H), 0.94 (d, J=6.6 Hz, 3H), 0.84 (d, J=6.6 Hz, 3H); ¹³C NMR (50.3 MHz, CDCl₃): δ=136.7, 135.6, 121.1, 116.8, 106.9, 68.4, 54.7, 54.4, 42.9, 40.2, 37.1, 36.0, 34.7, 27.0, 24.4, 22.2, 21.0, 17.1; IR (CCl₄): v=3590, 3480, 3064, 2945, 2919, 2820, 1516, 1457, 1438, 1380, 1361, 1155, 1105, 1065, 910; [α]_{D}²⁰=+63.6 (c=1.16, EtOAc); HRMS (ESI): m/z: calcd for C₁₈H₃₂NaO₃: 319.2249 (M+Na]⁺; found: 319.2241.

### EXAMPLE 2

tert-Butyl-[(R)-1-((1R,5R,6R)-6-dimethoxymethyl-5-isopropyl-2-methyl-cyclohex-2-enylmethyl)-but-3-enyloxy]-diphenyl-silane

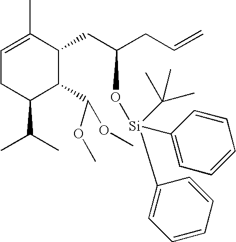

184 mg (0.62 mmol) of the compound prepared in example 1 were dissolved in 5 ml of dry CH₂Cl₂ and imidazole (211 mg, 3.11 mmol, 5 eq) is added. The solution was cooled to 0° C. and 318 μl (1.24 mmol, 2.0 eq) of TBDPSCl (tert-butyl-diphenyl-silylchloride) were slowly added. The reaction mixture was stirred at 0° C. for 90 min, then left stirring at room temperature over night, until complete reaction (TLC control). Then, the solvent was evaporated and the residue submitted to flash chromatography (Hex/EtOAc 25+1), R_{f}=0.4. Yield: 332 mg (0.62 mmol, quant.) of the tide compound as colourless oil.

R_{f}=0.40 (hexane/EtOAc 25:1); ¹H NMR (200 MHz, CDCl₃): δ=7.78-7.76 (m, 4H), 7.72-7.49 (m, 6H), 5.76-5.59 (m, 1H), 5.24 (m, 1H), 4.90-4.73 (m, 2H), 4.09 (d, J=5.6 Hz, 1H), 4.06-3.98 (m, 1H), 3.19 (s, 3H), 3.17 (s, 3H), 2.47-2.41 (m, 1H), 2.14-2.07 (m, 2H), 1.93-1.72 (m, 6H), 1.65 and 1.64 (s, 3H), 1.42-1.22 (m, 1H), 1.06 (s, 9H), 0.84 (d, J=6.8 Hz, 3H), 0.75 (d, J=6.6 Hz, 3H); ¹³C NMR (50.3 MHz, CDCl₃): δ=138.6, 136.1 (2C), 136.0 (2C), 135.3, 135.1, 134.8, 129.2 (2C), 127.3 (2C), 127.2 (2C), 120.5, 116.1, 107.6, 72.5, 54.7, 54.6, 42.3, 41.3, 38.1, 35.5, 35.1, 27.1, 27.1 (3C), 24.1, 22.9, 21.5, 19.4, 16.3; IR (CCl₄): v=3060, 3040, 2943, 2917, 2840, 1465, 1420, 1381 1320, 1105, 1075, 905; [α]_{D}²⁰=+46.4 (c=1.04, EtOAc); HRMS (ESI): m/z: calcd for C₃₄H₅₀NaO₃Si: 557.3427 [M+Na]⁺; found: 557.3413.

### EXAMPLE 3

(1R,2R,6R)-2-(R)-2-(tert-Butyl-diphenyl-silyloxy)-pent-4-enyl]-6-isopropyl-3-methyl-cyclohex-3-enecarbaldehyde.

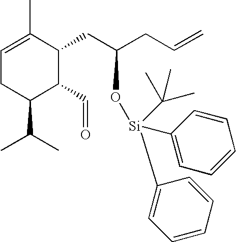

264 mg (0.49 mmol) of the compound prepared in example 2 were dissolved in 5 ml of a mixture of AcOH/H₂O/THF 3+1+1. The mixture was stirred at ambient temperature for 15 h until TLC analysis showed complete conversion (Hex/EtOAc 9+1, R_{f}=0.68). Ether, 5 g of NaHCO₃ and 10 ml of water were added and vigorous stirring was continued until no more gas evolves. The product was extracted with 3 portions of ether, the combined organic layers dried (Na₂SO₄), the solvent was distilled and pure title compound was obtained. Yield: 242 mg (0.49 mmol, quant.), colourless oil.

¹H NMR (200 MHz, CDCl₃): δ=9.55 (d, J=3.6 Hz, 1H), 7.71-7.58 (m, 4H), 7.48-7.29 (m, 6H), 5.72-5.51 (m, 1H), 5.30 (m, 1H), 4.99-4.79 (m, 2H), 3.79-3.69 (m, 1H), 2.43-1.01 (m, 22H), 0.88 (d, J=6.7 Hz, 3H), 0.74 (d, J=6.6 Hz, 3H); ³C NMR (50.3 MHz, CDCl₃): δ=206.5, 136.1, 135.9 (4C), 134.2, 134.1, 134.0, 129.7, 129.6, 127.6 (2C), 127.5 (2C), 121.6, 117.5, 72.1, 53.1, 41.6, 36.9, 35.7, 35.3, 27.1 (3C), 26.3, 23.8, 22.3, 20.6, 19.3, 16.9; IR (CCl₄): v=3060, 2945, 2918, 2842, 2700, 1713, 1465, 1458, 1420, 1382, 1363, 1105, 1061, 910; [α]_{D}²⁰=+28.4 (c=1.22, EtOAc); HRMS (ESI): m/z: calcd for C₃₂H₄₄NaO₂Si: 511.3008 [M+Na]⁺; found: 511.2996.

### EXAMPLE 4

{(1R,2R,6R)-2-[(R)-2-(tert-Butyl-dithenyl-silyloxy)-Pent-4-enyl]-6-isopropyl-3-methyl-cyclohex-3-enyl}-methanol

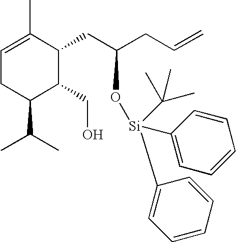

242 mg (0.49 mmol) of the compound prepared in example 3 were dissolved in 2 ml of EtOH. 28 mg (0.75 mmol, 1.5 eq) of NaBH₄ were added and the reaction mixture stirred at ambient temperature for 45 min. Then, another 10 mg (0.26 mmol, 0.5 eq) of NaBH₄ were added and stirring continued for 70 min. Then, 270 mg (5 mmol) of NH₄Cl were added, after 5 min, this mixture was diluted with ether. The ethereous phase was dried (Na₂SO₄), filtered over Celite, and the filtrate evaporated. The residue was purified by flash chromatography (Hex/EtOAc 4+1, R_{f}=0.31). Yield: 176 mg (0.358 mmol, 73%) of the title compound as colourless oil.

R_{f}=0.21 (hexane/EtOAc 25:1); ¹H NMR (200 MHz, CDCl₃): δ=7.77-7.57 (m, 4H), 7.45-7.34 (m, 6H), 5.92-5.75 (m, 1H), 5.26 (br s, 1H), 5.05-4.92 (m, 2H), 4.08-3.95 (m, 1H), 3.65 (dd, J=10.9, 5.6 Hz, 1H), 3.48-3.38 (m, 1H), 2.30 (t, J=5.8 Hz, 2H), 1.90-0.91 (m, 21H), 0.85 (d, J=6.8 Hz, 3H), 0.79 (d, J=6.8 Hz, 3H); ¹³C NMR (75.5 MHz, CDCl₃): δ=137.4, 136.0 (4C), 135.2, 134.7, 134.4, 129.6 (2C), 127.5 (4C), 121.5, 117.2, 72.3, 62.4, 41.7, 41.3, 36.9, 36.0, 34.7, 27.1 (4C), 24.1, 23.4, 21.2, 19.4, 16.2; IR (CCl₄): v=3615, 3060, 2945, 2918, 2880, 2842, 1422, 1381, 1363, 1105, 1058; [α]_{D}²⁰=+67.5 (c=0.99, EtOAc); HRMS (ESI): m/z: calcd for C₃₂H₄₇O₂Si: 491.3345 [M+H]⁺; found: 491.3341.

### EXAMPLE 5

Methanesulfonic acid (1R,2R,6R)-2-[(R)-2-(tert-butyl-diphenyl-silyloxy)-pent-4-enyl]-6-isopropyl-3-methyl-cyclohex-3-enylmethyl ester

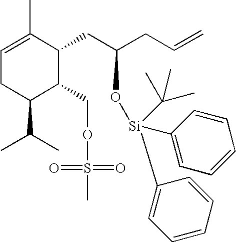

162 mg (0.33 mmol) of the compound prepared in example 4 were dissolved in 3 ml of dry CH₂Cl₂. The mixture was cooled to −40° C., 91 μl (67 mg, 0.66 mmol, 3 eq) of Et₃N and 40 μl (0.50 mmol, 1.5 eq) of MsCl (mesyl chloride) were added via syringe and the reaction was warmed to room temperature over 75 min. Then, NaHCO₃ sat. was added, the acqueous phase was extracted with 3 portions of CH₂Cl₂, the combined organic layers were dried over Na₂SO₄, the solvent was distilled and the product was purified by flash chromatography (Hex/EtOAc 4+1, R_{f}=0.63). Yield: 188 mg of the title compound (0.33 mmol, quant.) as colourless oil.

R_{f}=0.63 (hexane/EtOAc 4:1); ¹H NMR (200 MHz, CDCl₃): δ=7.65-7.59 (m, 4H), 7.44-7.30 (m, 6H), 5.88-5.67 (m, 1H), 5.23 (br s, 1H), 5.02-4.88 (m, 2H), 4.21 (dd, J=10.0, 6.5 Hz, 1H), 3.98 (dd, J=10.0, 8.5 Hz, 2H), 2.86 (s, 3H), 2.31-2.26 (m, 3H), 2.04-0.99. (m, 19H), 0.82 (d, J=6.0 Hz, 3H), 0.80 (d, J=6.0 Hz, 3H); “³C NMR (75.5 MHz, CDCl₃): δ=136.3, 135.9 (4C), 134.7, 134.4 (2C), 129.6 (2C), 127.6 (4C), 121.5, 117.5, 72.1, 70.1, 41.3, 39.0, 37.3, 37.0, 36.3, 34.8, 27.3, 27.1 (3C), 23.7, 23.2, 21.0, 19.4, 17.0; IR (CCl₄): v=3070, 2960, 2930, 2858, 1471, 1428, 1389, 1368, 1348, 1329, 1180, 1110, 1062; [α]_{D}²⁰=+58.1 (c=0.92, EtOAc); HRMS (ESI): m/z: calcd for C₃₃H₅₂NO₄SiS: 586.3386 [M+NH₄]⁺; found: 586.3368.

### EXAMPLE 6

{(1R,2R,6R)-2-[(R)-2-(tert-Butyl-diphenyl-silyloxy)-pent-4-enyl]-6-isopropyl-3-methyl-cyclohex-3-enyl}-acetonitrile.

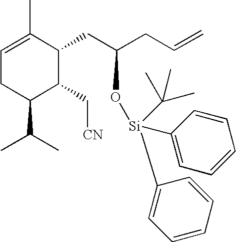

162 mg (285 μmol) of the compound prepared in example 5 were dissolved in 3 ml of dry acetonitrile. 56 mg (855 μmol, 3 eq) of KCN and 226 mg (855 μmol, 3 eq) of 18-crown-6 were added. The reaction mixture was heated to 80° C. for 6 h. The solvent was evaporated under reduced pressure, the residue was dissolved in Hex/EtOAc 9+1 and purified by flashchromatography (Hex/EtOAc 9+1, R_{f}=0.60), yield: 136 mg (272 μmol, 95%) of the title compound as colourless oil.

R_{f}=0.40 (hexane/EtOAc 14:1); ¹H NMR (200 MHz, CDCl₃): δ=7.71-7.69 (m, 4H), 7.47-7.33 (m, 6H), 5.84-5.70 (m, 1H), 5.22 (br s, 1H), 5.05-4.92 (m, 2H), 3.95-3.86 (m, 1H), 2.27-1.27 (m, 15H), 1.05 (s, 9H), 0.83 (d, J=6.8 Hz, 3H), 0.79 (d, J=6.8 Hz, 3H); ¹³C NMR (50.3 MHz, CDCl₃): δ=135.8 (4C), 135.4, 134.3, 134.2, 134.1, 129.5 (2C), 127.4 (4C), 121.2, 119.4, 117.6, 71.8, 40.9, 38.5, 36.3, 36.1, 35.9, 27.2, 27.0 (3C), 23.4, 22.8, 20.8, 19.2, 17.8, 17.5; IR (CCl₄): v=3070, 2955, 2922, 2890, 2856, 1715, 1470, 1460, 1425, 1388, 1369, 1110, 1070, 915; [α]_{D}²⁰=+66.4 (c=1.28, EtOAc); HRMS (ESI): m/z: calcd for C₃₃H₄₅NaNOSi: 522.3168 [M+Na]⁺; found: 522.3179.

### EXAMPLE 7

{(1R,2R,6R)-2-[(R)-2-(tert-Butyl-diphenyl-silyloxy)-pent-4-enyl]-6-isopropyl-3-methyl-cyclohex-3-enyl}-acetaldehyde.

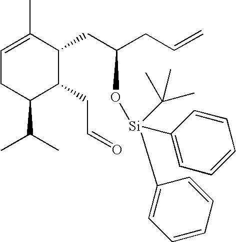

136 mg of the compound prepared in example 6 were dissolved in 3 ml of toluene/hexane 1+2. At −78° C., 2.7 ml (2.7 mmol, 10 eq) of DIBAlH (Di-isobutyl aluminium hydride, 1.0 M in hexane) were added. After stirring 45 min at −78° C. the reaction was quenched with 1.5 ml of EtOAc and 5 ml of 1 M tartaric acid. Extraction with 3 portions of CH₂Cl₂, washing the combined organic phases with 1N HCl, drying (Na₂SO₄) and purification by flash chromatography (Hex/EtOAc 9+1, R_{f}=0.60), 107 mg (121 mmol, 78%) of the title compound were obtained as colourless oil.

R_{f}=0.39 (hexane/EtOAc 14:1); ¹H NMR (200 MHz, CDCl₃): δ=9.66 (s, 1H), 7.78-7.71 (m, 4H), 7.457.35 (m, 6H), 5.91-5.71 (m, 1H), 5.26 (br s, 1H), 5.07-4.90 (m, 2H), 3.91 (q, J=5.9 Hz, 1H), 2.38-2.03 (m, 5H), 1.99-0.93 (m, 19H), 0.87 (d, J=6.7 Hz, 3H), 0.75 (d, J=6.7 Hz, 3H); ¹³C NMR (75.5 MHz, CDCl₃): δ=203.1, 136.6, 135.9 (4C), 134.6, 134.5, 134;3, 129.6 (2C), 127.5 (4C), 121.3, 117.4, 72.1, 44.2, 41.2, 38.9, 37.0, 36.4, 34.3, 27.4, 27.1 (3C), 23.7, 23.1, 21.2, 19.4, 17.2; IR (CCl₄): v=3060, 2943, 2917, 2880, 2842, 2695, 1720, 1465, 1455, 1420, 1382, 1365, 1103, 1095, 1060, 910; [α]_{D}²⁰=+53.3 (c=1.14, EtOAc).

### EXAMPLE 8

(R)-1-{(1R,2R,6R)-2-[(R)-2-(tert-Butyl-diphenyl-silyloxy)-pent-4-enyl]-6-isopropyl-3-methyl-cyclohex-3-enyl}-pent-4-en-2-ol.

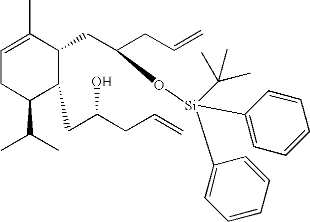

Allyl magnesium bromide (1.0 M in Et₂O, 150 μl, 0.15 mmol, 3 eq) was added to a solution of 170 μl (0.17 mmol, 3.3 eq) of ¹Ipc₂BOMe (¹Ipc derived from (−)-alpha-pinene, 1.0 M in THF) at 0° C. The mixture was stirred at room temperatur for 1 h. After 1 h, it was cooled to −78° C. and a solution of 25 mg (50 μmol) of the compound prepared in example 7 in 200 μl of dry ether were added. The mixture was stirred at −78° C. for 3 h 45 min, then it was warmed to room temperature and quenched with 200 μl of 30% H₂O₂ and 250 μl of 6M NaOH. Stirring was continued at ambient temperature for 40 min, water and ether were added and the product was isolated by extraction with 3 portions of ether, drying (Na₂SO₄) and intensive flash chromatography (Hex/EtOAc 4+1, R_{f}=0.59). Yield: 15 mg (28 μmol, 55%) of the title compound as a colourless oil.

R_{f}=0.59 (hexane/EtOAc 4:1); ¹H NMR (200 MHz, CDCl₃): δ=7.73-7.56 (m, 4H), 7.46-7.27 (m, 6H), 5.91-5.70 (m, 2H), 5.494.92 (m, 5H), 4.17-3.89 (m, 1H), 3.67-3.62 (m, 1H), 2.51-1.92 (m, 5H), 1.82-1.19 (m, 13H), 1.09 (s, 9H), 0.82 (d, J=6.7 Hz, 3H), 0.76 (d, J=6.7 Hz, 3H); ¹³C NMR (50.3 MHz, CDCl₃): δ□=136.9, 135.9 (4C), 135.0, 134.9, 134.6, 134.5, 129.5 (2C), 127.4 (4C), 121.1, 118.0, 117.0, 72.2, 68.3, 41.9, 41.5, 38.5, 37.1, 35.8, 35.4, 35.2, 27.1, 27.0 (3C), 23.9, 23.1, 21.2, 19.3, 17.5; IR (CCl₄): v=3595, 3078, 2960, 2934, 2899, 2860, 1642, 1475, 1465, 1430, 1389, 1371, 1110, 1069, 917; [α]_{D}²⁰=+38.8 (c=0.84, EtOAc); HRMS (ESI): m/z: calcd for C₃₆H₅₂NaO₂Si: 567.3634 [M+Na]⁺; found: 567.3665.

### EXAMPLE 9

Acetic acid (R)-1-{(1R,2R,6R)-2-[(R)-2-(tert-butyl-diphenyl-silyloxy)-pent-4-enyl]-6-isopropyl-3-methyl-cyclohex-3-enylmethyl}-but-3-enyl ester.

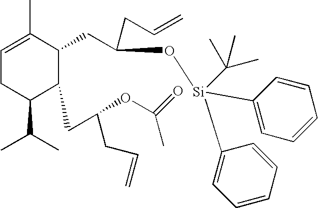

78 mg (0.14 mmol) of the compound prepared in example 8 were treated with acetic anhydride (41 μl, 0.44 mmol, 3 eq) and 4-DMAP (4-dimethylaminopyridine, 2 mg) in 500 μl of pyridine at 0° C. Stirring was continued until the reaction was complete by TLC (3 h). The mixture was diluted with EtOAc, washed with saturated KHSO₄ aqueous solution, dried (Na₂SO₄) and evaporated. The residue was purified by flash chromatography (Hexane/EtOAc 14+1, R_{f}=0.65). Yield: 75 mg (0.13 mmol, 93%) of the title compound as colourless oil.

R_{f}=0.58 (hexane/EtOAc 14:1); ¹H NMR (200 MHz, CDCl₃): δ=7.99-7.65 (m, 4H), 7.45-7.28 (m, 6H), 5.91-5.62 (m, 2H), 5.19-4.87 (m, 6H), 3.91 (q, J=5.7 Hz, 1H), 2.30-2.01 (m, 5H), 1.97 (s, 3H), 1.85-1.14 (m, 11H), 1.06 (s, 9H), 0.95-0.84 (m, 1H), 0.76 (d, J=6.6 Hz, 3H), 0.72 (d, J=6.6 Hz, 3H); ¹³C NMR (50.3 MHz, CDCl₃): δ=170.4, 135.8 (4C), 135.0, 134.5, 134.4 (2C), 133.6, 129.4 (2C), 127.3 (4C), 121.1, 117.6, 117.0, 71.9, 71.2, 41.4, 39.2, 39.0, 36.6, 34.8, 33.5, 31.5, 27.2, 27.0 (3C), 23.6, 22.9, 21.1, 20.9, 19.3, 19.2; IR (CCl₄): v=3050, 2934, 2911, 2831, 1726, 1460, 1450, 1415, 1375, 1359, 1096, 1090, 1052, 900; [α]_{D}²⁰=+27.9 (c=1.26, EtOAc); HRMS (ESI): m/z: calcd for C₃₈H₅₄NaO₃Si: 609.3740 [M+Na]⁺; found: 609.3746.

### EXAMPLE 10

11-Acetoxy-1-isopropyl-4-methyl-6-(tert-butyl-diphenyl-silyloxy)-1,2,4a,5,6,7,10,11,12,12a-decahydro-benzocyclodecene.

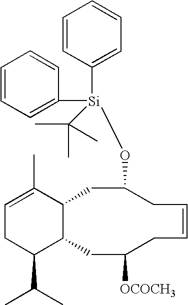

6 mg (10 μmol) of the compound prepared in example 9 was dissolved in 1 ml of dry degassed dichloromethane. 2 mg (2 μmol, 0.2 eq) of RCM catalyst A were dissolved in 500 μl of dry degassed dichloromethane and this solution was slowly added to the solution of the compound prepared in example 9 over 2 h under an argon atmosphere. The mixture was then stirred over night under an argon atmosphere. Another 1 mg (1 μmol, 0.1 eq) was dissolved in 250 μl of DCM and slowly added via syringe pump over 30 min. Stirring at ambient temperature was continued until no more starting material can be detected by TLC. Then, the solvent was distilled and the residue was submitted to flash chromatography (Hexane/EtOAc 9+1, R_{f}=0.53). Yield of the title compound: 5 mg (9 μmol, 90%).

R_{f}=0.43 (hexane/EtOAc 14:1); ¹H NMR (400 MHz, CDCl₃): δ=7.72-7.62 (m, 4H), 7.44-7.33 (m, 6H), 5.86 (dt, J=11.1, 4.7 Hz, 1H), 5.51 (ddd, J=11.7, 3.9 Hz, 1H), 5.20 (m, 1H), 5.13 (m, 1H), 4.17 (m, 1H), 2.77-2.49 (m, 2H), 2.29-2.25 (m, 1H), 2.15 (m, 1H), 2.05-0.85 (m, 23H), 0.89-0.85 (m, 1H), 0.81 (d, J=6.6 Hz, 3H), 0.77-0.65 (m, 1H), 0.61 (d, J=6.6 Hz, 3H); ¹³C NMR (50.3 MHz, CDCl₃): δ=170.4, 138.5, 135.7 (4C), 134.4 (2C), 129.5 (2C), 127.5 (4C), 126.9 (2C), 120.4, 72.8 (2C), 37.8, 37.0, 36.6, 34.7, 31.9, 31.6, 26.9 (4C), 26.2, 24.2, 23.9, 21.2, 20.9, 19.2, 14.9; IR (CCl₄): v=3070, 2958, 2926, 2856, 1740, 1460, 1427, 1368, 1110, 1067; [α]_{D}²⁰=+41.0 (c=1.26, EtOAc); HRMS [EI (70 eV)]: m/z: calcd for C₃₆H₅₀O₃Si: 558.3529 [M]⁺; found: 558.3532.

Assignment of the olefinic ³J_{cis} coupling constant (11.5 Hz between the protons at δ=5.51 and δ=5.86 ppm, respectively) by a 400 MHz H, H-COSY experiment.

Unequivocal assignment of cis stereochemistry of the double bond by detection of a NOE contact between olefinic protons in a 400 MHz NOESY experiment. 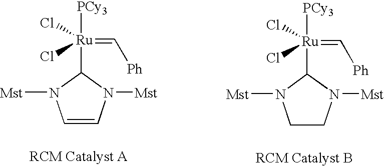
[MSt=C₆H₂-2,4,6-(CH₃)₃]

The same reaction was repeated with the RCM catalyst B (7% mol), in CH₂Cl₂ at room temperature, and after 168 h the title compound was isolated in 88% yield (95% after recovering starting material, stereochemistry of the double bond: 100% Z).

### EXAMPLE 11

11-Acetoxy-6-hydroxy-1-isopropyl-4-methyl-1,2,4a,5,6,7,10,11,12,12a-decahydro-benzocyclodecene.

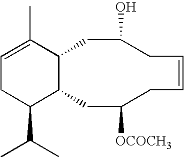

A solution of the compound prepared in example 10 (19 mg, 0.034 mmol) in THF (550 μl) was treated with 1 M TBAF in THF (170 μl, 5 eq.) at room temperature. The mixture was stirred for 16 h, then treated with pH 7 phosphate buffer, and extracted with EtOAc. The combined organic extracts were dried (Na₂SO₄) and evaporated to give a crude mixture, which was purified by flash-chromatography (hexane-EtOAc 4+1) to yield the title compound (10 mg, 94%).

R_{f}=0.27 (hexane/EtOAc 4:1); ¹H NMR (200 MHz, CDCl₃): δ=5.72 (dt, J=11.2, 3.9 Hz, 1H), 5.53 (dt, J=11.7, 4.0 Hz, 1H), 5.31-5.12 (m, 2H), 4.26-4.19 (m, 1H), 2.85-2.66 (m, 2H), 2.42-1.16 (m, 19H), 0.85 (d, J=6.8 Hz, 3H), 0.67 (d, J=6.8 Hz, 3H); ¹³C NMR (50.3 MHz, CDCl₃): δ=170.4, 138.4, 127.9, 126.2, 121.1, 72.6, 71.3, 38.0, 37.4, 36.8, 34.7, 31.9, 31.8, 27.0, 26.4, 24.4, 24.3, 21.2, 21.0, 15.1; IR (CCl₄): v=3634, 3621, 2924, 2843, 1739, 1460, 1382, 1364; [α]_{D}²⁰=+4.6 (c=0.46, EtOAc); HRMS (EST): m/z: calcd for C₂₀H₃₂NaO₃: 343.2249 [M+Na]⁺; found: 343.2241.

Preparation step A: (E)-3-(1H-Imidazol-4-yl)-2-propenoic Acid Ethyl Ester.

(E)₃-(1H-Imidazol-4-yl)-2-propenoic Acid (2.0 g, 14.48 mmol) was dissolved in absolute EtOH (10 mL) under N₂. H₂SO₄ (conc., 1 ml) was added and the mixture was refluxed for 3 h until the solution was clear. The mixture was neutralized with saturated aqueous NaHCO₃ solution (pH=7). The solution was extracted with EtOAc (3×20 mL) and the combined organic layers dried over Na₂SO₄. The solvent was evaporated under reduced pressure to yield the title compound (2.19 g, 13.19 mmol, 91%) as a white solid.

¹H-NMR (CDCl₃, 200 MHz): δ=1.31 (t, J=7.1 Hz, 3H, CO₂CH₂CH₃), 4.22 (q, J=7.1 Hz, 2H, CO₂CH₂CH₃), 6.44 (d, J=15.7 Hz, 1H, CH═CH—CO₂CH₂CH₃), 7.29 (s, 1H, Ar—H), 7.60 (d, J=15.7 Hz, 1H, CH═CH—CO₂CH₂CH₃), 7.71 (s, 1H, Ar—H), 8.30 (br s, 1H, NH).

Preparation Step B (E)-3-(1-Methyl-Imidazol-4-yl)-2-propenoic Acid Ethyl Ester

The compound prepared in preparation step A (2.19 g, 13.19 mmol) was dissolved in absolute CH₃CN (20 ml) under N₂. K₂CO₃ (1.0 eq., 13.19 mmol, 1.82 g) and MeI (1.0 eq., 13.19 mmol, 1.87 g) were added. The solution stirred for 24 h at room temperature in a closed flask (TLC showed starting material left). MeI (0.5 eq., 6.60 mmol, 0.91 g) were added and the solution stirred for another 48 h at room temperature in a closed flask (TLC showed minimal starting material left). CHCl₃ (20 mL) and H₂O (20 mL) were added. The layers were separated and the aqueous layer was extracted with CHCl₃ (2×10 mL). The combined organic layers were dried over Na₂SO₄. The solvent was evaporated under reduced pressure and the resulting residue was purified by flash chromatography (CH₂Cl₂/EtOH 97:3) to yield the title compound (714 mg, 3.96 mmol, 30%) as a white solid. R_{f}=0.55 (CH₂Cl₂/EtOH 97:3); ¹H-NMR (CDCl₃, 200 MHz): δ=1.28 (t, J=7.1 Hz, 3H, CO₂CH₂CH₃), 3.68 (s, 3H, N—CH₃), 4.21 (q, J=7.1 Hz, 2H, CO₂CH₂CH₃), 6.50 (d, J=15.7 Hz, 1H, CH═CH—CO₂CH₂CH₃), 7.05 (s, 1H, Ar—H), 7.42 (s, 1H, Ar—H), 7.51 (d, J=15.7 Hz, 1H, CH═CH—CO₂CH₂CH₃).

Preparation step C (E)-3-(1-Methyl-Imidazol-4-yl)-2-propenoic Acid Sodium Salt

The compound prepared in preparation step B (187 mg, 1.038 mmol) was dissolved in THF/H₂O (1:1, 8 ml). NaOH (1.0 eq., 1.038 mmol, 41.5 mg) was added and the solution stirred 48 h at room temperature (TLC showed minimal starting material left and the pH was neutral, between 7-8). The solvent was evaporated under reduced pressure, coevaporated with benzene (5×5 mL) and dried at a high vacuum pump to yield the title compound as a grey-white solid, that was used without further purification.

¹H-NMR (DMSO-D₆, 200 MHz): δ=3.60 (s, 3H, N—CH₃), 6.20 (d, J=15.7 Hz, 1H, CH═CH—CO₂CH₂CH₃), 6.96 (d, J=15.7 Hz, 1H, CH═CH—CO₂CH₂CH₃), 7.16 (s, 1H, Ar—H), 7.50 (s, 1H, Ar—H).

### EXAMPLE 12

3-(1-Methyl-1H-imidazol-4-yl)-acrylic acid 11-acetoxy-1-isopropyl-4-methyl-1,2,4a,5,6,7,10,11,12,12a-decahydro-benzocyclodecen-6-yl ester.

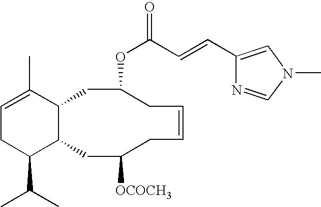

The compound prepared in the preparation step C (15.0 eq., 0.515 mmol, 90 mg) was dissolved in dry THF (5 mL) under Ar. Pivaloylchloride (15.0 eq, 0.515 mmol, 62 mg) was added and the solution stirred for 24 h at room temperature in a closed flask. The solution was filtered under Ar to separate the precipitated NaCl, the precipitate was washed with dry THF (2×5 mL) and the solvent carefully evaporated under reduced pressure to give the corresponding crude mixed anhydride as a white suspension (checked by ¹H-NMR in CDCl₃). The alcohol prepared in example 11 (1.0 eq., 0.0343 mmol, 11.0 mg) was dissolved in dry CH₂Cl₂ (2 ml) and added to the solution of the crude mixed anhydride under Ar. NEt₃ (15.0 eq., 0.515 mmol, 52 mg) and DMAP (1.0 eq., 0.0343 mmol, 4 mg) were added and the the solution stirred for 3 d at room temperature. The solvent was evaporated under reduced pressure and the resulting residue was purified by flash chromatography (Hexane/EtOAc 1:4) to yield the title compound (9.2 mg, 0.020 mmol, 59%) as an oil.

R_{f}=0.25 (hexane/EtOAc 1:4); ¹H NMR (400 MHz, CDCl₃): δ=7.55 (d, J=15.6 Hz, 1H), 7.47 (s, 1H), 7.09 (s, 1H), 6.54 (d, J=15.6 Hz, 1H), 5.74-5.66 (m, 1H), 5.62-5.55 (m, 1H), 5.45-5.39 (m, 1H), 5.34 (br s, 1H), 5.245.18 (m, 1H), 3.72 (s, 3H), 2.88-2.77 (m, 2H), 2.40-2.25 (m, 2H), 2.232.18 (m, 1H), 2.07 (s, 3H), 2.05-1.26 (m, 12H), 0.87 (d, J=6.7 Hz, 3H), 0.70 (d, J=6.7 Hz, 3H); ¹³C NMR (50.3 MHz, CDCl₃): δ=170.4, 166.7, 137.9, 138.4, 135.9, 127.9, 126.4, 122.2, 121.0, 116.4, 73.6, 72.5, 37.5, 37.3, 34.6, 33.5, 32.7, 31.6, 29.6, 29.2, 26.9, 26.2, 24.3, 24.2, 21.2, 20.9, 15.1; IR (CCl₄): v=2960, 2850, 1745, 1705, 1640, 1450, 1440, 1380, 1345, 1295, 905; [α]_{D}²⁰=−29 (c=0.71, EtOAc); HRMS [EI (30 eV)]: m/z: calcd for C₂₇H₃₈N₂O₄: 454.2832 [M]⁺; found: 454.2802.

### EXAMPLE 13

(4R,4aR,6R,11R,12aR)-11-(tert-Butyl-diphenyl-silanoxy)-4-isopropyl-1-methyl-3,4,4a,5,6,7,10,11,12,12a-decahydro-benzocyclodecen-6-ol

The compound prepared in Example 10 (30 mg, 0.05 mmol) was dissolved in MeOH (1 mL). K₂CO₃ (17 mg, 0.10 mmol) was added and the solution stirred for 15 h at room temperature. H₂O (2 mL) was added, the layers were separated and the aqueous layer was extracted with EtOAc (3×5 mL), the combined organic layers were washed with a saturated aqueous NaCl solution (2×5 mL) and the combined organic layers were dried over Na₂SO₄. The solvent was evaporated under reduced pressure and the resulting residue was purified by flash chromatography (hexane/EtOAc 9:1) to provide the title compound (26 mg, 94%) as a colourless oil. R_{f}=0.25 (hexane/EtOAc 9:1); ¹H NMR (200 MHz, CDCl₃): δ=7.74-7.61 (m, 4H), 7.46-7.31 (m, 6H), 5.82 (dt, J=11.3, 5.1 Hz, 1H), 5.52 (dt, J=11.3, 5.1 Hz, 1H), 5.13 (br s, 1H), 4.23-4.04 (m, 2H), 2.70-2.49 (m, 2H), 2.33-2.04 (m, 3H), 1.99-1.18 (m, 13H), 1.08 (s, 9H), 0.84 (d, J=6.8 Hz, 3H), 0.72 (d, J=6.8 Hz, 3H); ¹³C NMR (50.3 MHz, CDCl₃): δ=138.5, 135.7 (4C), 134.5, 134.4, 129.4 (2C), 127.9, 127.5 (4C), 126.6, 120.4, 72.7, 71.0, 37.9, 37.3, 36.6; 35.5, 33.7, 32.0, 29.1, 27.0 (4C), 24.3, 23.9, 21.0, 19.2, 15.3; IR (CCl₄): v=3611, 2942, 2921, 2842, 1472, 1458, 1427, 1389, 1369, 1109, 1067, 909; [α]_{D}²⁰=+25.3 (c=0.73, EtOAc); HRMS (ESI): m/z: calcd for C₃₄H₄₈NaO₂Si: 539.3321 [M+Na]⁺; found: 539.3306.

### EXAMPLE 14

(4R,4aR,11R,12aR)-11-(tert-Butyl-diphenyl-silanoxy)-4-isopropyl-1-methyl-3,4a,5,7,10,11,12,12a-octahydro-4H-benzocyclodecen-6-one:

(COCl)₂ (91 mg, 0.72 mmol) was dissolved in CH₂Cl₂ (0.5 mL) and cooled to −60° C. DMSO (77 mg, 0.98 mmol) was added and the solution stirred for 10 min at −60° C.

The compound prepared in Example 13 (62 mg, 0.12 mmol) in CH₂Cl₂ (0.5 mL) was added and the solution stirred for further 30 min at −60° C. NEt₃ (199 mg, 1.97 mmol) was added, the solution was allowed to warm to 0° C. over 1 h and stirred additional 10 min at 0° C. An aqueous phosphate buffer solution (3 mL, pH=7) was added, the layers were separated and the aqueous layer was extracted with CH₂Cl₂ (3×5 mL) and the combined organic layers were dried over Na₂SO₄. The solvent was evaporated under reduced pressure and the resulting residue was purified by flash chromatography (hexane/EtOAc 9:1) to provide unreacted starting compound (9 mg) and the title compound (50 mg, 81%, 95% after recovering starting material) as colourless oils. R_{f}=0.55 (hexane/EtOAc 9:1);

¹H NMR (200 MHz, CDCl₃): δ=7.77-7.59 (m, 4H), 7.47-7.29 (m, 6H), 6.19-6.06 (m, 1H), 5.97-5.84 (m, 1H), 5.06 (br s, 1H), 3.96-3.82 (m, 1H), 3.20-2.87 (m, 3H), 2.32-1.95 (m, 4H), 1.89-1.61 (m, 5H), 1.41-1.18 (m, 5H), 1.07 (s, 9H), 0.87 (d, J=6.8 Hz, 3H), 0.69 (d, J=6.8 Hz, 3H); ¹³C NMR (50.3 MHz, CDCl₃): δ=213.0, 139.2, 135.8 (4C), 134.0 (2C), 131.4, 129.7, 129.6, 127.6 (2C), 127.5 (2C), 123.7, 116.7, 73.0, 44.1, 38.7, 36.0, 36.7 (2C), 36.5, 32.1, 27.0 (3C), 26.6, 24.1, 22.6, 21.1, 19.2, 14.3, IR (CCl₄): v=3022, 2955, 2922, 2899, 2860, 1708, 1705, 1469, 1427, [α]_{D}²⁰=+49.0 (c=0.79, EtOAc); HRMS (ESI): m/z: calcd for C₃₄H₄₆NaO₂Si: 537.3165 [M+Na]⁺; found: 537.3139.

### EXAMPLE 15

(4R,4aR,11R,12aR)-11-Hydroxy-4-isopropyl-1-methyl-3,4a,5,7,10,11,12,12a-octahydro-4H-benzocyclodecen-6-one

The compound prepared in example 14 (16 mg, 0.03 mmol) was dissolved in THF (0.50 mL) and TBAF (0.06 mL, 0.06 mmol, 1.0 M in THF) was added. The reaction mixture stirred 23 h at room temperature. Additional TBAF (0.06 mL, 0.06 mmol, 1.0 M in THF) was then added and the solution stirred for further 5 h at room temperature. An aqueous phosphate buffer solution (1.0 mL, pH=7) was added, the layers were separated and the aqueous layer was extracted with EtOAc (3×5 mL) and the combined organic layers were dried over Na₂SO₄. The solvent was evaporated under reduced pressure and the residue was purified by flash chromatography (hexane/EtOAc 8:2) to yield the title compound (10 mg, quant.) as a colourless oil. R_{f}=0.17 (hexane/EtOAc 8:2); ¹H NMR (200 MHz, CDCl₃): δ=6.01-5.84 (m, 2H), 5.23 (br s, 1H), 4.07-3.93 (m, 1H), 3.24-2.87 (m, 3H), 2.42-2.08 (m, 4H), 1.92-1.01 (m, 11H), 0.87 (d, J=6.9 Hz, 3H), 0.71 (d, J=6.9 Hz, 3H); ¹³C NMR (75.5 MHz, CDCl₃): δ=212.8, 139.0, 130.3, 124.6, 119.5, 71.9, 44.2, 39.0, 38.0, 37.1, 37.0, 36.9, 32.1, 26.6, 24.2, 23.3, 21.2, 14.4; IR (CCl₄): v=3627, 2960, 2951, 2904, 1709, 1460, 1442, 1389, 1371, 909; [α]_{D}²⁰=−4.7 (c=0.51, EtOAc).

### EXAMPLE 16

(E)-3-(1-Methyl-1H-imidazol-4-yl)-acrylic acid [(1R,4aR,6R,12aR)-1-isopropyl-4-methyl-11-oxo-1,2,4a,5,6,7,10,11,12,12a-decahydro-benzocyclodecen-6-enyl]ester

The compound prepared in Example 15 (9 mg, 0.03 mmol) was dissolved in CH₂Cl₂ (2 mL) and added to Mixed Anhydride (110 mg, 0.47 mmol). NEt₃ (48 mg, 0.47 mmol) and DMAP (4 mg, 0.03 mmol) were added and the solution stirred for 3 d at 40° C. Thel solvent was evaporated under reduced pressure and the resulting residue was purified by flash chromatography (hexane/EtOAc 1:5) to yield the title compound (7 mg, 52%) as a colourless oil. R_{f}=0.18 (hexane/EtOAc 1:5); ¹H NMR (400 MHz, CDCl₃): δ=7.55 (d, J=15.6 Hz, 1H), 7.47 (s, 1H), 7.09 (s, 1H), 6.55 (d, J=15.6 Hz, 1H), 5.97-5.88 (m, 2H), 5.26 (br s, 1H), 5.15 (d, J=12.0 Hz, 1H), 3.72 (s, 3H), 3.25-3.15 (m, 1H), 3.09-2.96 (m, 2H), 2.46-2.14 (m, 4H), 1.94-1.55 (m, 7H), 1.48-1.17 (m, 3H), 0.89 (d, J=6.9 Hz, 3H), 0.75 (d, J=6.9 Hz, 3H); ¹³C NMR (100.8 MHz, CDCl₃): δ=212.7, 166.6, 139.1, 139.0. 138.6, 136.0, 130.6, 124.7, 122.3, 119.2, 116.3, 73.7, 44.1, 39.1, 37.0, 36.8 (2C), 34.1, 33.5, 29.7, 26.7, 24.2, 23.1, 21.1, 14.4; IR (CCl₄): v=2945, 2920, 2890, 1703, 1640, 1452, 1381, 1390, 1153, 1104, 905; [α]_{D}²⁰=−15.6 (c=0.34, EtOAc); HRMS (ESI): m/z: calcd for C₂₅H₃₅N₂O₃: 411.2648 [M+H]⁺; found: 411.2648.

### EXAMPLE 17

(1R,4aR,6R,12aR)-tert-Butyl-(1-isopropyl-4-methyl-11-methylene-1,2,4a5,6,7,10,11,12,12a-decahydro-benzocyclodecen-6-yloxy)-diphenyl-silane

Ph₃PCH₃Br (17 mg, 0.047 mmol) was dissolved in THF (0.2 mL). n-BuLi (13 μL, 0.020 mmol, 1.6 M in n-hexane) was added and the solution stirred for 1 h at room temperature. The compound prepared in Example 14 (8 mg, 0.016 mmol) was added in THF (600 μL) and the solution was heated to 50° C. for 12 h. H₂O (2.0 mL) was added and the layers were separated. The aqueous layer was extracted with EtOAc (3×5 mL) and the combined organic layers were dried over Na₂SO₄. The solvent was evaporated under reduced pressure and the residue was purified by flash chromatography (hexane) to yield the title compound (8 mg, 94%) as a colourless oil. R_{f}=0.32 (hexane); ¹H NMR (200 MHz, CDCl₃): δ=7.72-7.65 (m, 4H), 7.46-7.33 (m, 6H), 6.03-5.87 (m, 1H), 5.73-5.61 (m, 1H), 5.11 (br s, 1H), 4.94 (br s, 1H), 4.75 (br s, 1H), 4.04-3.93 (m, 1H), 3.08 (dd, J=16.1, 5.9 Hz, 1H), 2.84-2.70 (m, 2H), 2.19-1.44 (m, 8H), 1.41-1.17 (m, 6H), 1.09 (s, 9H), 0.88 (d, J=5.8 Hz, 3H), 0.75 (d, J=5.8 Hz, 3H); ¹³C NMR (50.3 MHz, CDCl₃): δ=138.4, 135.9 (4C), 134.5 (2C), 133:8, 129.6, 129.5, 128.8, 128.6 (2C), 127.6, 127.5 (2C), 119.2, 111.1, 73.3, 39.4, 37.7, 37.3, 37.1, 36.3, 32.1, 30.7, 27.1 (3C), 26.6, 24.5, 23.1, 21.1 (2C), 19.3; IR (CCl₄): v=3070, 3018, 2955, 2925, 2856, 1470, 1459, 1423, 1385, 1366, 1308, 1108, 1071; [α]_{D}²⁰=+68.4 (c=0.25, EtOAc).

### EXAMPLE 18

(1R,4aR,6R,12aR)-1-Isopropyl-4-methyl-11-methylene-1,2,4a,5,6,7,10,11,12,12a-decahydro-benzocyclodecen-6-ol

The compound prepared in Example 17 (8 mg, 0.015 mmol) was dissolved in THF (1.0 mL) and TBAF (73 μL, 0.075 mmol, 1.0 M in THF) was added. The reaction mixture stirred 12 h at room temperature. Additional TBAF (73 μL, 0.075 mmol, 1.0 M in THF) was added and the reaction mixture stirred for another 12 h at room temperature. An aqueous phosphate buffer solution (1.0 mL, pH=7) was added, the layers were separated and the aqueous layer was extracted with EtOAc (3×5 mL) and the combined organic layers were dried over Na₂SO₄. The solvent was evaporated under reduced pressure and the residue was purified by flash chromatography (hexanes/EtOAc 9:1) to yield the title compound (4 mg, quant.) as a colourless oil. R_{f}=0.28 (hexanes/EtOAc 9:1); ¹H NMR (200 MHz, CDCl₃): δ=5.76-5.62 (m, 2H), 5.26 (br s, 1H), 4.98 (s, 1H), 4.80 (s, 1H), 4.08-3.97 (m, 1H), 3.24-3.12 (m, 1H), 2.98-2.74 (m, 2H), 2.29-1.67 (m, 9H), 1.61-1.34 (m, 5H), 0.87 (d, J=6.7 Hz, 4H), 0.77 (d, J=6.7 Hz, 3H); ¹³C NMR (50.3 MHz, CDCl₃): δ=133.8, 129.8, 127.0, 123.3, 120.0, 111.7, 72.0, 39.7, 37.7, 37.5, 36.9, 36.7, 32.2, 31.1, 26.8, 24.6, 23.6, 21.1 (2C); IR (CC14): V=3632, 3080, 3024, 2967, 2930, 1461, 1455, 1440, 1389, 1370; [α]_{D}²⁰=+36.4 (c=0.17, EtOAc).

### EXAMPLE 19

(E)-3-(1-Methyl-1H-imidazol-4-yl)-acrylic acid [(1R,4aR,6R,12aR)-(1-isopropyl-4-methyl-11-methylene-1,2,4a5,6,7,10,11,12,12a-decahydro-benzocyclodecen-6-yl]ester

The compound prepared in Example 18 (4 mg, 0.0091 mmol) was dissolved in CH₂Cl₂ (1.0 mL) and added to Mixed Anhydride (51 mg, 0.137 mmol). NEt₃ (22 mg, 0.219 mmol) and DMAP (1 mg, 0.008 mmol) were added and the solution stirred for 3 d at room temperature. The solvent was evaporated under reduced pressure and the resulting residue was purified by flash chromatography (hexane/EtOAc 1:4) to yield the title compound (3 mg, 47%) as a colourless oil. R_{f}=0.30 (hexane/EtOAc 1:4); ¹H NMR (200 MHz, CDCl₃): δ=7.62 (br s, 1H), 7.54 (d, J=15.7 Hz, 1H), 7.08 (br s, 1H), 6.59 (d, J=15.7 Hz, 1H), 5.74-5.62 (m, 2H), 5.30-5.11 (m, 2H), 5.04 (br s, 1H), 4.72 (br s, 1H), 3.73 (s, 3H), 3.25-2.73 (m, 3H), 2.34-1.55 (m, 7H), 1.441.21 (m, 6H), 0.87 (d, J=6.7 Hz, 4H), 0.77 (d, J=6.7 Hz, 3H); ¹³C NMR (50.3 MHz, CDCl₃): δ=164.3, 148.2, 138.2, 135.4, 131.5, 131.3, 129.6, 127.6, 119.7, 113.8, 111.6, 74.7, 39.3, 37.4, 36.9, 36.2, 34.8, 33.7, 30.9, 29.7, 29.5, 26.5, 24.5, 23.4, 21.0 (2C); IR (CCl₄): v=3380, 3075, 2956, 2922, 2856, 1766, 1709, 1645, 1460, 1428, 1382, 1368, 1305, 1159, 1099; [α]_{D}²⁰=+6.0 (c=0.05, EtOAc); HRMS (ESI): m/z: calcd for C₂₆H₃₇N₂O₂: 409.2855 [M+H]⁺; found: 409.2855.

### EXAMPLES 20 AND 21

(2S,3S)-1-{(1R,2R,6R)-2-[(2R)-2-(tert-Butyl-diphenyl-silanyloxy)-pent-4-enyl]-6-isopropyl-3-methyl-cyclohex-3-enyl}-3-methoxymethoxy-Dent4-en-2-ol (20) and (2R,3R)-1-{(1R,2R,6R)-2-[(2R)-2-(tert-Butyl-diphenyl-silanyloxy)-pent-4-enyl]-6-isopropyl-3-methyl-cyclohex-3-enyl}-3-methoxymethoxy-pent-4-en-2-ol (21)

Methoxymethyl allyl ether (180 mg, 1.75 mmol) in THF (4.0 mL) was cooled to −780C and sec-BuLi (1.10 mL, 1.45 mmol, 1.3 M in cyclohexane) was added. The reaction solution was stirred at −78° C. for 30 min and ¹Ipc₂BOMe (1.60 mL, 1.45 mmol, 0.93 M in THF) was then added. Stirring was maintained for 1 h, BF₃*Et₂O (263 mg, 1.86 mmol) was then added, followed by the compound prepared in example 7 (293 mg, 0.58 mmol) in THF (2.0 mL). The mixture was stirred at −78° C. for 5 h and then slowly warmed to room temperature. An aqueous NaOH solution (4.0 mL, 6.0 M) and H₂O₂ (4.0 mL, 35%) were then added and the mixture left stirring overnight. H₂O (5 mL) was added and the aqueous layer was extracted with EtOAc (3×10 mL) and the combined organic layers were dried over Na₂SO₄. The solvent was evaporated under reduced pressure and the resulting residue was purified by flash chromatography (hexane/EtOAc 6:1) to provide unreacted starting compound (56 mg), the title compound 20 (246 mg, 70%) and 21 (25 mg, 7%) as colourless oils (total yield 77%, 84% after recovering starting material, de 82%, 20:21 10:1). 20: R_{f}=0.43 (hexane/EtOAc 6:1); ¹H NMR (200 MHz, CDCl₃): δ=7.95-7.66 (m, 4H), 7.45-7.30 (m, 6H), 5.91-5.51 (m, 2H), 5.32-5.16 (m, 3H), 5.01-4.90 (m, 2H), 4.72 (d, J=6.7 Hz, 1H), 4.56 (d, J=6.7 Hz, 1H), 3.95 (q, J=5.9 Hz, 1H), 3.79-3.72 (m, 1H), 3.58-3.47 (m, 1H), 3.38 (s, 3H), 2.35-2.14 (m, 5H), 1.93-1.14 (m, 11H), 1.05 (s, 9H), 0.79 (d, J=6.6 Hz, 3H), 0.77 (d, J=6.6 Hz, 3H); ¹³C NMR (50.3 MHz, CDCl₃): μ=136.4, 135.9 (4C), 135.1, 134.9, 134.7, 134.6, 129.4 (2C), 127.4 (4C), 120.9, 119.9, 117.0, 93.9, 81.9, 72.2, 71.4, 55.7, 41.3, 39.3, 37.1, 35.8, 34.3, 31.3, 27.5, 27.1 (3C), 23.9, 22.9, 21.2, 19.3, 19.2; IR (CCl₄): v=3590, 3078, 2955, 2893, 2860, 1473, 1462, 1430, 1390, 1369, 1152, 1108, 935, 915; [α]_{D}²⁰=+43.8 (c=0.90, EtOAc); HRMS (ESI): m/z: calcd for C₃₈H₅₆NaO₄Si: 627.3845 [M+Na]⁺; found: 627.3828; 21: R_{f}=0.37 (hexane/EtOAc 6:1); ¹H NMR (200 MHz, CDCl₃): δ=7.71-7.60 (m, 4H), 7.47-7.30 (m, 6H), 5.96-5.77 (m, 1H), 5.65-5.49 (m, 1H), 5.38-5.21 (m, 2H), 5.16 (br s, 1H), 5.07-4.92 (m, 2H), 4.73 (d, J=6.7 Hz, 1H), 4.58 (d, J=6.7 Hz, 1H), 3.98-3.83 (m, 1H), 3.81-3.69 (m, 1H), 3.65-3.50 (m, 1H), 3.39 (s, 3H), 2.46-1.95 (m, 4H), 1.90-0.98 (m, 20H), 0.90-0.87 (m, 1H), 0.83 (d, J=6.8 Hz, 3H), 0.69 (d, J=6.8 Hz, 3H); ¹³C NMR (50.3 MHz, CDCl₃): δ=137.3, 135.9 (4C), 135.1, 134.8, 134.7 (2C), 129.4 (2C), 127.4 (4C), 121.9, 120.2, 117.0, 93.7, 82.4, 72.1, 70.3, 55.8, 40.8, 37.5, 37.1, 35.8, 35.4, 31.3, 29.7, 27.0 (3C), 26.8, 24.3, 23.9, 21.1, 19.3; IR (CCl₄): v=3600, 3080, 2960, 2938, 2899, 2862, 1475, 1432, 1391, 1372, 1158, 1108, 912; [α]_{D}²⁰+48.1 (c=1.09, EtOAc); HRMS (ESI): m/z: calcd for C₃₈H₅₆NaO₄Si: 627.3846 [M+Na]⁺; found: 627.3860.

### EXAMPLE 22

2,2-Dimethyl-propionic acid {(1S,2S)-1-{(1R,2R,6R)-2-[(2R)-2-(tert-butyl-diphenyl-silanyloxy)-pent-4-enyl]-6-isopropyl-3-methyl-cyclohex-3-enylmethyl}-2-methoxymethoxy-but-3-envyl}ester.

The compound prepared in Example 20 (173 mg, 0.29 mmol) was dissolved in pyridine (2.0 mL). DMAP (4 mg, 0.03 mmol) and PivCl (172 mg, 1.43 mmol) were added. The mixture stirred at room temperature for 72 h. EtOAc (10 mL) were added, the organic layer was washed with a saturated aqueous KHSO₄ solution (2×10 mL), the combined aqueous layers were back extracted with EtOAc (3×10 mL) and the combined organic layers were dried over Na₂SO₄ The solvent was evaporated under reduced pressure and the resulting residue was purified by flash chromatography (hexane/EtOAc 9:1) to provide the title compound (158 mg, 80%) as a colourless oil. R_{f}=0.52 (hexane/EtOAc 9:1); ¹H NMR (300 MHz, CDCl₃): , =7.71-7.66 (m, 4H), 7.45-7.28 (m, 6H), 5.85-5.59 (m, 2H), 5.29-5.17 (m, 3H), 5.03-4.90 (m, 3H), 4.65 (d, J=6.3 Hz, 1H), 4.56 (d, J=6.3 Hz, 1H), 4.09-3.87 (m, 2H), 3.35 (s, 3H), 2.30-2.12 (m, 3H), 1.85 (br s, 1H), 1.72-1.22 (m, 11H), 1.18 (s, 9H), 1.04 (s, 9H), 0.82 (d, J=6.3 Hz, 3H), 0.72 (d, J=6.3 Hz, 3H); ¹³C NMR (50.3 MHz, CDCl₃): δ=177.6, 135.9 (4C), 135.3, 134.8, 134.6 (2C), 134.1, 129.5 (2C), 127.5 (4C), 121.0, 119.0, 117.2, 94.4, 77.8, 72.2, 71.7, 55.6, 41.4, 39.4, 36.8, 34.8, 32.7, 27.4, 27.3 (3C), 27.2, 27.0 (3C), 23.6, 22.5, 21.0, 20.6, 19.3 (2C); IR (CCl₄): v=3061, 2942, 2920, 2882, 2842, 1722, 1455, 1467, 1458, 1423, 1392, 1385, 1362, 1150, 1107, 1098, 909; [α]_{D}²⁰=+25.5 (c=0.78, EtOAc); HRMS (ESI): m/z: calcd for C₄₃H₆₄NaO₅Si: 711.4420 [M+Na]⁺; found: 711.4412.

### EXAMPLE 23

2,2-Dimethyl-propionic acid {(1S,2S)-1-{(1R,2R,6R)-2-[(2R)-2-(tert-butyl-diphenyl-silanoxy)-pent-4-enyl]-6-isopropyl-3-methyl-cyclohex-3-enylmethyl]-2-hydroxy-but-3-enyl}ester.

The compound prepared in Example 22 (10 mg, 0.015 mmol) was dissolved in CH₂Cl₂ (0.3 mL) and cooled to −78° C. PhSH (2 mg, 0.015 mmol) and BF₃*Et₂O (4 mg, 0.029 mmol) were added and the solution was allowed to warm to −100C and stirred at −10° C. for 2 h. Then a saturated aqueous NaHCO₃ solution (2 mL) was added and the solution was warmed up to room temperature. The layers were separated, the aqueous layer was extracted with EtOAc (3×5 mL) and the combined organic layers were dried over Na₂SO₄. The solvent was evaporated under reduced pressure and the resulting residue was purified by flash chromatography (hexane/EtOAc 8:2) to provide the title compound (6 mg, 64%) as a colourless oil. R_{f}=0.54 (hexane/EtOAc 8:2); ¹H NMR (200 MHz, CDCl₃): δ=7.71-7.67 (m, 4H), 7.41-7.31 (m, 6H), 5.89-5.72 (m, 2H), 5.34 (br s, 1H), 5.25-5.14 (m, 2H), 5.03-4.91 (m, 3H), 4.08 (br s, 1H), 3.93-3.87 (m, 1H), 2.27-2.16 (m, 3H), 2.01-1.22 (m, 14H), 1.18 (s, 9H), 1.04 (s, 9H), 0.93-0.88 (m, 1H), 0.82 (d, J=6.4 Hz, 3H), 0.72 (d, J=6.4 Hz, 3H); “³C NMR (50.3 MHz, CDCl₃): δ=176.1, 137.1 (2C), 136.4, 135.9 (2C), 135.0 (2C), 134.7, 129.5 (2C), 127.4 (4C), 121.0, 117.2, 116.4, 74.4, 73.4, 72.3, 41.4, 39.2, 36.7, 35.0, 33.3, 28.0, 27.5, 27.3 (3C), 27.0 (3C), 23.8, 22.6, 20.9 (2C), 20.0, 19.3 (2C); 1R (CCl₄): v=3618, 3582, 3060, 2942, 2918, 2842, 1724, 1472, 1465, 1458, 1421, 1382, 1363, 1149, 1101, 1060, 908; [α]_{D}²⁰=+6.1 (c=0.56, EtOAc); HRMS (ESI): m/z: calcd for C₄₁H₆₂NaO₄Si: 669.4315 [M+Na]⁺; found: 669.4299.

### EXAMPLE 24

2,2-Dimethyl-propionic acid [(4R,4aR,6S,7S,11R,12aR)-11-(tert-butyl-diphenyl-silanoxy)-7-hydroxy-4-isoproyl-1-methyl-3,4,4a,5,6,7,10,11,12,12a-decahydro-benzocyclodecen-6-yl]ester

The compound prepared in Example 23 (33 mg, 0.05 mmol) was dissolved in degassed CH₂Cl₂ (5.1 mL). Grubbs-II (2 mg, 2.5 μmol) in degassed CH₂Cl₂ (0.5 mL) was slowly added. The reaction mixture stirred for 2 d at room temperature. Additional Grubbs-II (1 mg, 1.3 μmol) in degassed CH₂Cl₂ (0.2 mL) was slowly added and the mixture stirred for further 2 d at room temperature. Additional Grubbs-II (1 mg, 1.3 μmol) in degassed CH₂Cl₂ (0.2 mL) was slowly added and the mixture stirred for further 1 d at room temperature. The solvent was evaporated under reduced pressure and the residue was purified by flash chromatography (hexane/EtOAc 8:2) to provide the title compound (23 mg, 73%) as a colourless oil. R_{f}=0.44 (hexane/EtOAc 8:2); ¹H NMR (400 MHz, CDCl₃): δ=7.73-7.65 (m, 4H), 7.47-7.35 (m, 6H), 5.99 (dt, J=11.5, 5.2 Hz, 1H), 5.58 (t, J=10.5 Hz, 1H), 5.14 (br s, 1H), 5.05-4.96 (m, 1H), 4.77 (t, J=9.9 Hz, 1H), 4.23-4.16 (m, 1H), 2.65-2.57 (m, 1H), 2.32-2.27 (m, 1H), 1.89-1.54 (m, 9H), 1.40-1.05 (m, 23H), 0.84 (d, J=6.8 Hz, 3H), 0.64 (d, J=6.8 Hz, 3H); ¹³C NMR (50.3 MHz, CDCl₃): δ=178.1, 137.1, 135.8 (4C), 135.4, 134.8, 134.5 (2C), 129.4 (2C), 127.4 (4C), 121.0, 117.2, 116.4, 74.4, 73.3, 72.2, 41.3, 39.2, 36.6, 34.6, 33.1, 27.7, 27.3 (3C), 26.9 (3C), 23.6, 22.6, 20.9 (2C), 20.0; IR (CCl₄): v=3630, 2958, 2925, 2852, 1730, 1425, 1367, 1155, 1112, 908; [α_{D}=+17.5 (c=0.60, EtOAc); HRMS (ESI): m/z: calcd for C₃₉H₅₆NaO₄Si: 639.3846 [M+Na]⁺; found: 639.3855.

### EXAMPLE 25

2,2-Dimethyl-propionic acid [(4R,4aR,6S,7S,1R,12aR)-11-(tert-butyl-diphenyl-silanyloxy)-4-isopropyl-7-methoxy-1-methyl-3,4,4a5,6,7,10,11,12,12a-decahydro-benzocyclodecen-6-yl}ester

The compound prepared in Example 24 (14 mg, 0.023 mmol) was dissolved in CH₂Cl₂ (0.2 mL). 2,6-Di-tert-butyl-pyridine (44 mg, 0.23 mmol) and MeOTf (19 mg, 0.12 mmol) were added and the solution stirred for 18 h at 40° C. The solvent was evaporated under reduced pressure and the resulting residue was purified by flash chromatography (hexane/EtOAc 9:1) to provide the title compound (14 mg, 96%) as a colourless oil. R_{f}=0.28 (hexane/EtOAc 9:1); ¹H NMR (200 MHz, CDCl₃): δ=7.74-7.60 (m, 4H), 7.54-7.27 (m, 6H), 6.08 (dt, J=11.4, 5.3 Hz, 1H), 5.40 (t, J=10.7 Hz, 1H), 5.16-5.01 (m, 2H), 4.29-4.08 (m, 2H), 3.18 (s, 3H), 2.68-2.49 (m, 1H), 2.35-2.17 (m, 1H), 2.09-0.84 (m, 31H), 0.80 (d, J=6.8 Hz, 3H), 0.57 (d, J=6.8 Hz, 3H); ³C NMR (75.5 MHz, CDCl₃): δ=177.7, 138.6, 135.8 (4C), 134.3 (2C), 130.9, 129.6 (2C), 129.2, 127.6 (4C), 120.5, 78.8, 74.8, 72.2, 56.2, 37.6, 37.1, 36.4, 35.3, 32.6, 29.7, 27.2 (3C), 27.0 (3C), 26.9, 26.7, 24.3, 24.1, 21.0 (2C), 19.2; IR (CCl₄): v=2948, 2920, 2848, 1723, 1475, 1422, 1385, 1363, 1279, 1155, 1099, 1059; [α]_{D}²⁰=+7.8 (c=0.77, EtOAc); HRMS (ESI): m/z: calcd for C₄₀H₅₈NaO₄Si: 653.4002 [M+Na]⁺; found: 653.3986.

### EXAMPLE 26

2,2-Dimethyl-propionic acid [(4R,4aR,6S,7S,11R,12aR)-11-hydroxy-4-isopropyl-7-methoxy-1-methyl-3,4,4a,5,6,7,10,11,12,12a-decahydro-benzocyclodecen-6-yl]ester.

The compound prepared in Example 25 (14 mg, 0.022 mmol) was dissolved in THF (0.50 mL) and TBAF (0.044 mL, 0.044 mmol, 1.0 M in THF) was added. The reaction mixture stirred 14 h at room temperature. An aqueous phosphate buffer solution (1.0 mL, pH=7) was added, the layers were separated and the aqueous layer was extracted with EtOAc (3×5 mL) and the combined organic layers were dried over Na₂SO₄. The solvent was evaporated under reduced pressure and the residue was purified by flash chromatography (hexanes/EtOAc 8:2) to yield the title compound (8 mg, 89%) as a colourless oil. R_{f}=0.11 (hexanes/EtOAc 8:2); ¹H NMR (400 MHz, CDCl₃): i=5.87 (dt, J=11.6, 5.4 Hz, 1H), 5.44 (t, J=10.8 Hz, 1H), 5.32 (br s, 1H), 5.12-5.08 (m, 1H), 4.34-4.23 (m, 2H), 3.26 (s, 3H), 2.80-2.74 (m, 1H), 2.48-2.2.31 (m, 1H), 2.25-1.12 (m, 20H), 1.05-0.89 (m, 3H), 0.86 (d, J=6.8 Hz, 3H), 0.64 (d, J=6.8 Hz, 3H); ¹³C NMR (100.8 MHz, CDCl₃): δ=177.6, 137.5, 130.0, 129.5, 121.0, 78.3, 74.7, 70.7, 56.2, 37.8, 37.3, 36.9, 36.6, 35.3, 32.4, 29.6, 27.2 (3C), 26.9, 26.7, 24.4, 21.0 (2C); IR (CCl₄): v=3620, 2954, 2923, 2864, 1728, 1478, 1451, 1395, 1386, 1367, 1280, 1160, 1099, 905; [α]_{D}²⁰=−8.4 (c=0.37, EtOAc).

### EXAMPLE 27

(E)-3-(1-Methyl-1H-imidazol-4-yl)-acrylic acid [(1R,4aR,6R,10S,11S,12aR)-11-(2,2-dimethyl-propionyloxy)-1-isopropyl-10-methoxy-4-methyl-1,2,4a,5,6,7,10,11,12,12a-decahydro-benzocyclodecen-6-yl}ester

The compound prepared in Example 26 (6 mg, 0.0153 mmol) was dissolved in CH₂Cl₂ (1.8 mL) and added to Mixed Anhydride (76 mg, 0.325 mmol). NEt₃ (33 mg, 0.325 mmol) and DMAP (2 mg, 0.0153 mmol) were added and the solution stirred for 15 d at room temperature. The solvent was evaporated under reduced pressure and the resulting residue was purified by flash chromatography (hexane/EtOAc 1:5) to yield the title compound (6 mg, 66%) as a colourless oil. R_{f}=0.22 (hexane/EtOAc 1:5); ¹H NMR (400 MHz, CDCl₃): δ=7.56 (d, J=15.6 Hz, 1H), 7.48 (s, 1H), 7.10 (s, 1H), 6.53 (d, J=15.6 Hz, 1H), 5.96-5.89 (m, 1H), 5.52-5.38 (m, 2H), 5.32 (br s, 1H), 5.12 (d, J=7.3 Hz, 1H), 4.27 (t, J=9.9 Hz, 1H), 3.73 (s, 3H), 3.26 (s, 3H), 2.88-2.76 (m, 1H), 2.542.46 (m, 1H), 2.13-1.14 (m, 24H), 0.99-0.87 (m, 2H), 0.85 (d, J=5.1 Hz, 3H), 0.65 (d, J=5.1 Hz, 3H); ¹³C NMR (100.8 MHz, CDCl₃): δ=177.7, 166.6, 139.2, 138.5, 136.1, 135.7, 130.3, 129.6, 122.3, 121.1, 116.3, 79.7, 74.7, 72.9, 56.7, 37.4 (2C), 35.3, 33.5, 33.2, 30.7, 29.6, 29.3, 27.2 (3C), 26.9, 26.6, 24.3, 24.4, 21.0 (2C); IR (CCl₄): v=2960, 2931, 2889, 2861, 1731, 1712, 1645, 1481, 1460, 1389, 1300, 1157, 1103; [α]_{D}²⁰=−17.0 (c=0.33, EtOAc); HRMS (ESI): m/z: calcd for C₃₂H₅₀NaN₂O₅: 565.3617 [M+Na]⁺; found: 565.3611.

### EXAMPLE 28

2,2-Dimethyl-propionic acid [(4R,4aR,6S,7S,11R,2aR)-7-acetoxy-1-(tert-butyl-diphenyl-silanyloxy)-4-isopropyl-1-methyl-3,4,4a5,6,7,10,11,12,12a-decahydro-benzocyclodecen-6-yl]ester

The compound prepared in Example 24 (16 mg, 0.026 mmol) was dissolved in pyridine (0.5 mL). Ac₂O (5 mg, 0.052 mmol) and DMAP (cat.) were added and the reaction mixture stirred for 24 h at room temperature. EtOAc (5 mL) was added and the organic layer was washed with a saturated aqueous KHSO₄ solution (2×5 mL), the aqueous layer was back extracted with EtOAc (3×5 mL) and the combined organic layers were dried over Na₂SO₄. The solvent was evaporated under reduced pressure and the residue was purified by flash chromatography (hexane/EtOAc 9:1) to provide the title compound (12 mg, 71%) as a colourless oil. R_{f}=0.69 (hexaneJEtOAc 9:1); ¹H NMR (200 MHz, CDCl₃): δ=7.69-7.61 (m, 4H), 7.46-7.30 (m, 6H), 6.11-5.87 (m, 2H), 5.45 (t, J=10.7 Hz, 1H), 5.22-5.10 (m, 2H), 4.24-4.18 (m, 1H), 2.88-2.74 (m, 1H), 2.32-2.25 (m, 1H), 2.12-1.95 (m, 4H), 1.84-0.89 (m, 30H), 0.82 (d, J=6.9 Hz, 3H), 0.57 (d, J=6.9 Hz, 3H); “³C NMR (75.5 MHz, CDCl₃): δ=177.5, 170.0, 138.8, 135.7 (4C), 134.1 (2C), 132.1, 129.6, 129.5, 127.6 (2C), 127.5 (2C), 126.5, 120.3, 73.6, 72.0, 71.5, 37.7, 37.3, 36.4, 35.3, 32.9, 29.6, 27.04 (7C), 26.5, 24.3, 24.0 (2C), 21.0, 19.2, 14.5; IR (CCl₄): v=3078, 2959, 2938, 2860, 1739, 1732, 1482, 1460, 1430, 1371, 1155, 1112, 1070; [α]_{D}²⁰=+0.6 (c=0.51, EtOAc); HRMS (ESI): m/z: calcd for C₄₁H₅₈NaO₅Si: 681.3951 [M+Na]⁺; found: 681.3967.

### EXAMPLE 29

2,2-Dimethyl-propionic acid [(4R,4aR,6S,7S,11R,12aR)-7-acetoxy-11-hydroxy-4-isopropyl-1-methyl-3,4,4a,5,6,7,10,11,12,12a-decahydro-benzocyclodecen-6-yl]ester.

The compound prepared in Example 28 (11 mg, 0.0167 mmol) was dissolved in THF (0.30 mL) and TBAF (0.033 mL, 0.0334 mmol, 1.0 M in THF) was added. The reaction mixture stirred 20 h at room temperature. An aqueous phosphate buffer solution (1.0 mL, pH=7) was added, the layers were separated and the aqueous layer was extracted with EtOAc (3×5 mL) and the combined organic layers were dried over Na₂SO₄. The solvent was evaporated under reduced pressure and the residue was purified by flash chromatography (hexanes/EtOAc 8:2) to yield the title compound (7 mg, 92%) as a colourless oil. R_{f}=0.15 (hexanes/EtOAc 8:2); ¹H NMR (200 MHz, CDCl₃): δ=6.01 (t, J=10.3 Hz, 1H), 5.91 (dt, J=11.4, 5.4 Hz, 1H), 5.47 (t, J=10.7 Hz, 1H), 5.29 (br s, 1H), 5.19 (dd, J=10.1, 6.9 Hz, 1H), 4.46-4.24 (m, 1H), 3.08-2.91 (m, 1H), 2.44-2.33 (m, 1H), 2.18-1.22 (m, 17H), 1.18 (s, 9H), 0.84 (d, J=6.8 Hz, 3H), 0.61 (d, J=6.8 Hz, 3H); “³C NMR (75.5 MHz, CDCl₃): δ=177.4, 171.1, 138.8, 130.9, 127.3, 120.9, 73.5, 71.3, 70.5, 37.9, 37.4, 36.6, 35.3, 32.7, 29.7, 27.1 (3C), 26.9, 26.4, 24.4, 21.0 (2C), 14.5; IR (CCl₄): v=3622, 2957, 2935, 2870, 1745, 1730, 1479, 1452, 1395, 1388, 1369, 1280, 1155; [α]_{D}²⁰=−33.5 (c=0.39, EtOAc); HRMS (ESI): m/z: calcd for C₂₅H₄₀NaO₅: 443.2773 [M+Na]⁺; found: 443.2761.

### EXAMPLE 30

(E)-3-(1-Methyl-1H-imidazol-4-yl)-acrylic acid [(1R,4aR,6R,10S,11S,12aR)-10-acetoxy-11-(2,2-dimethyl-propionyloxy)-1-isopropyl-4-methyl-1,2,4a5,6,7,10,11,12,12a-decahydro-benzocyclodecen-6-yl]ester

The compound prepared in Example 29 (6 mg, 0.014 mmol) was dissolved in CH₂Cl₂ (1.6 mL) and added to Mixed Anhydride (50 mg, 0.21 mmol). NEt₃ (21 mg, 0.21 mmol) and DMAP (2 mg, 0.014 mmol) were added and the solution stirred for 2 d at room temperature. The solvent was evaporated under reduced pressure and the resulting residue was purified by flash chromatography (hexane/EtOAc 1:4) to yield the title compound (6 mg, 80%) as a colourless oil. R_{f}=0.20 (hexane/EtOAc 1:4); ¹H NMR (400 MHz, CDCl₃): δ=7.60 (s, 1H), 7.54 (d, J=15.7 Hz, 1H), 7.11 (s, 1H), 6.59 (d, J=15.7 Hz, 1H), 6.04 (t, J 10.4 Hz, 1H), 5.89 (dt, J=11.6, 5.6 Hz, 1H), 5.55-5.45 (m, 2H), 5.32 (s, 1H), 5.23 (dd, J=10.1, 6.8 Hz, 1H), 3.75 (s, 3H), 3.13-3.03 (m, 1H), 2.54-2.48 (m, 1H), 2.22-1.17 (m, 25H), 0.85 (d, J=6.9 Hz, 3H), 0.63 (d, J=6.9 Hz, 3H); ¹³C NMR (75.5 MHz, CDCl₃): δ=177.6, 170.1, 166.7, 138.6, 138.4, 136.1, 130.9, 127.5 (2C), 122.3, 120.9, 116.3, 73.4, 72.8, 71.1, 37.5, 36.5, 35.3, 33.7, 33.5, 30.0, 29.7, 27.2 (3C), 26.8, 26.3, 24.5, 21.0 (3C), 14.5; IR (CCl₄): v=2961, 2935, 1742, 1733, 1712, 1648, 1390, 1371, 1155; [α]_{D}²⁰==39.6 (c=0.24, EtOAc); HRMS (ESI): m/z: calcd for C₃₂H₄₇N₂O₆: 555.3434 [M+H]⁺; found: 555.3425.

### EXAMPLE 31

2,2-Dimethyl-propionic acid {(4R,4aR,6S,7S,11R,12aR)-11-(tert-butyl-diphenyl-silanyloxy)-4-isopropyl-1-methyl-7-[(2′*)-tetrahydro-pyran-2′-yloxy)1-3,4,4a5,6,7,10,11,12,12a-decahydro-benzocyclodecen-6-yl}ester.

The compound prepared in Example 24 (29 mg, 0.047 mmol) was dissolved in CH₂Cl₂ (0.4 mL). Dihydropyran (6 mg, 0.071 mmol) and PPTS (1 mg, 0.005 mmol) were added and the solution stirred for 11 h at room temperature. i-Pr₂O (5 mL) was added, the organic layer was washed with a semisaturated NaCl solution (2×5 mL) and the organic layer was dried over Na₂SO₄. The solvent was evaporated under reduced pressure and the resulting residue was purified by flash chromatography (hexane/EtOAc 14:1) to yield the title compound (25 mg, 77%) as a colourless oil. R_{f}=0.33 (hexane/EtOAc 14:1); ¹H NMR (250 MHz, CDCl₃, signal doubling due to diastereomers): δ=7.68-7.61 (m, 4H), 7.38-7.28 (m, 6H), 6.08+5.93 (dt, J=11.6, 5.5 Hz, 1H), 5.59+5.32 (t, J=10.4 Hz, 1H), 5.18-5.01 (m, 2H), 4.88-4.61 (m, 2H), 4.23-4.10 (m, 1H), 3.94-3.72 (m, 1H), 3.51-3.33 (m, 1H), 2.76-2.57 (m, 1H), 2.35-2.17 (m, 1H), 2.13-1.95 (m, 1H), 1.91-0.95 (m, 36H), 0.80 (d, J=5.8 Hz, 3H), 0.61-0.55 (m, 3H); ³C NMR (50.3 MHz, CDCl₃, signal doubling due to diastereomers): δ=177.6+177.4, 138.9+138.8, 135.7 (4C), 134.4+134.2 (2C), 132.0, 130.0+129.5 (2C), 128.4, 127.9+127.5+127.4 (4C), 120.2, 99.2+93.0, 76.5, 74.9+74.6, 72.1+70.5, 61.5+60.4, 37.7, 37.3, 36.5, 35.4, 32.6, 30.4+30.1, 29.6, 27.3 (3C), 27.0+26.6 (3C), 26.3, 25.4, 24.3, 23.9, 21.0 (2C), 19.2, 16.7+16.3, 14.6; IR (CCl₄): v=3062, 2945, 2918, 2844, 1722, 1474, 1465, 1448, 1422, 1381, 1362, 1151, 1107, 1059, 902; [α]_{D}²⁰−7.2 (c=1.16, EtOAc); HRMS (ESI): m/z: calcd for C₄₄H₆₄NaO₅Si: 723.4421 [M+Na]⁺; found: 723.4416.

### EXAMPLE 32

2,2-Dimethyl-propionic acid {(4R,4aR,6S,7S,11R,12aR)-11-hydroxy-4-isopropyl-1-methyl-[(2′*)-7-(tetrahydro-pyran-2′-yloxy)]-3,4,4a,5,6,7,10,11,12,12a-decahydro-benzocyclodecen-6-yl}ester.

The compound prepared in Example 31 (23 mg, 0.033 mmol) was dissolved in THF (2.0 mL) and TBAF (165 μL, 0.165 mmol, 1.0 M in THF) was added. The reaction mixture 30 stirred 20 h at room temperature. An aqueous phosphate buffer solution (1.0 mL, pH=7) was added, the layers were separated and the aqueous layer was extracted with EtOAc (3×5 mL) and the combined organic layers were dried over Na₂SO₄. The solvent was evaporated under reduced pressure and the residue was purified by flash chromatography (hexanes/EtOAc 4:1) to yield the title compund (15 mg, quant.) as a colourless oil. R_{f}=0.10 (hexanes/EtOAc 4:1); ¹H NMR (200 MHz, CDCl₃, signal doubling due to diastereomers): δ=5.97+5.78 (dt, J=9.3, 5.8 Hz, 1H), 5.60+5.35 (t, J=10.3 Hz, 1H), 5.29-5.01 (m, 2H), 4.93-4.65 (m, 2H), 4.39-4.15 (m, 1H), 4.02-3.73 (m, 1H), 3.55-3.33 (m, 1H), 2.93-2.71 (m, 1H), 2.40-2.22 (m, 1H), 2.17-1.09 (m, 29H), 0.82 (d, J=6.7 Hz, 3H), 0.62+0.59 (d, J=6.7 Hz, 3H); ¹³C NMR (100.8 MHz, CDCl₃, signal doubling due to diastereomers): δ=177.5, 133.8, 129.2, 126.6, 120.9+120.8, 99.3+93.1, 76.5, 74.9+74.7, 70.7+70.4, 61.5+60.6, 37.9+37.8, 37.4, 36.6, 35.5, 32.4, 30.4+30.2, 27.4, 27.3 (3C), 27.2, 26.9, 26.7+26.3, 25.5+25.4, 24.5, 21.1 (2C), 18.7+18.4; IR (CCl₄): v=3620, 3440, 2950, 2865, 1723, 1477, 1451, 1392, 1385, 1365, 1279, 1155, 1110, 1072, 1050, 905; [α]_{D}²⁰=−36.1 (c=0.75, EtOAc); HRMS (ESI): m/z: calcd for C₂₈H₄₆NaO₅: 485.3243 [M+Na]⁺; found: 485.3238.

### EXAMPLE 33

3-(1-Methyl-1H-imidazol-4-yl)-acrylic acid {(1R,4aR,6R,10S,11S,12aR)-11-(2,2-dimethyl-propionyloxy)-1-isopropyl-4-methyl-10-[(2′*-(tetrahydro-pyran-2-yloxy)]-1,2,4a,5,6,7,10,11,12,12a-decahydro-benzocyclodecen-6-yl}ester.

The compound prepared in Example 32 (15 mg, 0.032 mmol) was dissolved in CH₂Cl₂ (2.0 mL) and added to Mixed Anhydride (118 mg, 0.494 mmol). NEt₃ (50 mg, 0.494 mmol) and DMAP (4 mg, 0.032 mmol) were added and the solution stirred for 3 d at room temperature. The solvent was evaporated under reduced pressure and the resulting residue was purified by flash chromatography (hexane/EtOAc 1:4) to yield the title compound (12 mg, 61%) as a colourless oil. R_{f}=0.15 (hexane/EtOAc 1:4); ¹H NMR (400 MHz, CDCl₃, signal doubling due to diastereomers): δ=7.62 (s, 1H), 7.57+7.53 (d, J=15.7 Hz, 1H), 7.10 (s, 1H), 6.56 (d, J=15.7 Hz, 1H), 5.94+5.75 (dt, J=9.1, 6.2 Hz, 1H), 5.65 (t, J=10.4 Hz, 1H), 5.45-5.37 (m, 2H), 5.30 (s, 1H), 5.23-5.08 (m, 1H), 4.92+4.78 (t, J=10.2 Hz, 1H), 4.90-4.85+4.68-4.65 (m, 1H), 3.99-3.92+3.84-3.77 (m, 1H), 3.74 (s, 3H), 3.57-3.47+3.45-3.38 (m, 1H), 2.97-2.83 (m, 1H), 2.48-2.41 (m, 1H), 2.17-1.38 (m, 1SH), 1.33-1.18 (m, 12H), 0.84 (d, J=6.8 Hz, 3H), 0.67-0.61 (m, 3H); ¹³C NMR (50.3 MHz, CDCl₃, signal doubling due to diastereomers): δ=177.2, 166.5, 137.9, 135.2, 130.9+130.8, 129.4, 126.7, 122.1, 120.8, 117.0, 99.3+93.3, 74.7+74.5, 73.1, 70.2, 61.5+60.8, 37.4, 36.6, 35.4, 33.7, 30.3, 29.6, 27.3 (3C), 27.0, 26.8, 26.5, 26.2, 25.4, 24.5, 24.3, 21.0 (2C), 19.0+18.7, 14.6; IR (CCl₄): v=2959, 2877, 1725, 1709, 1645, 1481, 1452, 1385, 1398, 1156, 1113, 909; [α]_{D}²⁰=−44.4 (c=0.59, EtOAc); HRMS (ESI): m/z: calcd for C₃₂H₅₃N₂O₆: 597.3904 [M+H]⁺; found: 597.3900.

### EXAMPLE 34

3-(1-Methyl-1H-imidazol-4-yl)-acrylic acid [(1R,4aR,6R,10S,10S,12aR)-11-(2,2-dimethyl-propionyloxy)-10-hydroxy-1-isopropyl-4-methyl-1,2,4a,5,6,7,10,11,12,12a-decahydro-benzocyclodecen-6-yl}ester.

The compound prepared in Example 33 (13 mg, 0.022 mmol) was dissolved in EtOH (2.0 mL, 80%). PTSA (0.8 mg, 0.0044 mmol) were added and the solution stirred for 72 h at room temperature. EtOAc (5 mL) was added, the organic layer was washed with a saturated aqueous NaHCO₃ solution (2×5 mL), with a saturated aqueous NaCl solution (5 mL) and then the organic layer was dried over Na₂SO₄. The solvent was evaporated under reduced pressure and the resulting residue was purified by flash chromatography (EtOAc) to yield the title compound (7 mg, 82%) as a colourless oil. R_{f}=0.16 (EtOAc); ¹H NMR (400 MHz, CDCl₃): δ=7.67 (s, 1H), 7.54 (d, J=5.2 Hz, 1H), 7.09 (s, 1H), 6.58 (d, J=5.2 Hz, 1H), 5.77 (dt, J=11.5, 5.3 Hz, 1H), 5.60 (t, J=10.5 Hz, 1H), 5.43-5.37 (m, 1H), 5.29 (s, 1H), 5.11-5.00 (m, 1H), 4.84 (t, J=9.8 Hz, 1H), 3.74 (s, 3H), 2.87-2.78 (m, 1H), 2.51-1.71 (m, 11H), 1.62-1.36 (m, 3H), 1.35-1.15 (m, 10H), 0.84 (d, J=6.8 Hz, 3H), 0.64 (d, J=6.8 Hz, 3H); ¹³C NMR (100.8 MHz, CDCl₃): δ=178.5, 166.5, 138.9, 137.7, 135.1, 131.4, 128.2, 122.4, 121.1, 117.2, 77.7, 73.1, 70.4, 69.5, 37.4, 33.9, 33.1, 30.1, 29.7, 27.3 (3C), 27.2, 26.9, 26.8, 24.4, 24.3, 21.0 (2C), 19.1; IR (CCl₄): v=3620, 3240, 2957, 2922, 2865, 1709, 1643, 1478, 1455, 1385, 1367, 1322, 1297, 1155, 1109, 1043, 908; [α]_{D}²⁰=−45.0 (c=0.28, EtOAc); HRMS (ESI): m/z: calcd for C₃₀H₄₅N₂O₅: 513.3329 [M+H]⁺; found: 513.3321.

### EXAMPLE 35

2,2-Dimethyl-propionic acid {(1R,2R)-{(1R,2R,6R)-2-[(2R)-2-(tert-butyl-diphenyl-silanyloxy)-pent-4-enyl]-6-isopropyl-3-methyl-cyclohex-3-enylmethyl}-2-methoxymethoxy-but-3-enyl}ester.

The compound prepared in Example 21 (26 mg, 0.043 mmol) was dissolved in pyridine (1.0 mL). DMAP (1 mg, 0.004 mmol) and PivCl (26 mg, 0.215 mmol) were added. The mixture stirred at room temperature for 72 h. EtOAc (5 mL) were added, the organic layer was washed with a saturated aqueous KHSO₄ solution (2×5 mL), the combined aqueous layers were back extracted with EtOAc (3×5 mL) and the combined organic layers were dried over Na₂SO₄ The solvent was evaporated under reduced pressure and the resulting residue was purified by flash chromatography (hexane/EtOAc 9:1) to provide the title compound (27 mg, 91%) as a colourless oil. R_{f}=0.71 (hexane/EtOAc 9:1); ¹H NMR (200 MHz, CDCl₃): δ=7.67-7.59 (m, 4H), 7.42-7.28 (m, 6H), 6.03-5.91 (m, 1H), 5.87-5.76 (m, 1H), 5.31-4.95 (m, 6H), 4.63 (d, J=6.7 Hz, 1H), 4.55 (d, J=6.7 Hz, 1H), 4.04-3.89 (m, 2H), 3.35 (s, 3H), 2.23-2.15 (m, 3H), 1.96-1.91 (m, 2H), 1.87-0.80 (m, 28H), 0.75 (d, J=6.7 Hz, 3H), 0.62 (d, J=6.7 Hz, 3H); ¹³C NMR (50.3 MHz, CDCl₃): δ=173.7, 137.4, 135.9 (4C), 135.1, 134.9, 134.6, 134.3, 129.4, 129.3, 127.4 (2C), 127.3 (2C), 121.8, 119.2, 117.2, 94.3, 78.5, 71.8, 71.5, 55.7, 40.3, 37.2, 37.0, 35.8, 35.6, 29.7, 28.8, 27.3 (3C), 27.1 (4C), 24.3, 23.8, 21.0 (2C), 19.3; IR (CCl₄): v=2935, 2905, 2867, 2830, 1728, 1431, 1372, 1159, 1107, 910; [α_{D}²⁰=+52.7 (c=0.77, EtOAc); HRMS (ESI): m/z: calcd for C₄₃H₆₄NaO₅Si: 711.4421 [M+Na]⁺; found: 711.4406.

### EXAMPLE 36

2,2-Dimethyl-propionic acid {(1R,2R)-1-{(1R,2R,6R)-2-[(2R)-2-(tert-butyl-diphenyl-silanoxy)-pent-4-4-enyl]-6-isopropyl-3-methyl-cyclohex-3-enylmethyl}-2-hydroxy-but-3-enyl}ester.

The compound prepared in Example 35 (32 mg, 0.046 mmol) was dissolved in CH₂Cl₂ (0.5 mL) and cooled to −78° C. PhSH (10 mg, 0.093 mmol) and BF₃*Et₂O (13 mg, 0.093 mmol) were added and the solution was allowed to warm to −10° C. over 3 h and stirred at −10° C. for 2 h. PhSH (5 mg, 0.046 mmol) and BF₃*Et₂O (7 mg, 0.046 mmol) were added again and the solution stirred at −10° C. for another 1 h. Then a saturated aqueous NaHCO₃ solution (1 mL) was added and the solution was warmed up to room temperature. The layers were separated, the aqueous layer was extracted with EtOAc (3×5 mL) and the combined organic layers were dried over Na₂SO₄. The solvent was evaporated under reduced pressure and the resulting residue was purified by flash chromatography (hexane/EtOAc 9:1) to provide the title compound (19 mg, 63%) as a colourless oil. R_{f}=0.40 (hexane/EtOAc 9:1); ¹H NMR (200 MHz, CDCl₃): δ=7.74-7.63 (m, 4H), 7.48-7.28 (m, 6H), 6.05-5.68 (m, 2H), 5.36-4.88 (m, 6H), 4.08-3.89 (m, 2H), 2.41-2.18 (m, 2H), 2.09-1.95 (m, 1H), 1.84-0.82 (m, 31H), 0.78 (d, J=6.7 Hz, 3H), 0.66 (d, J=6.7 Hz, 3H); ¹³C NMR (75.5 MHz, CDCl₃): δ=178.3, 137.3, 137.1, 135.9 (4C), 135.1, 134.6 (2C), 129.5 (2C), 127.4 (4C), 121.7, 117.3, 116.3, 74.3, 73.1, 71.6, 40.5, 37.4, 36.7, 35.6, 35.3, 29.7, 28.7, 27.2 (3C), 27.1 (4C), 24.0, 23.7, 21.0 (2C), 19.3; IR (CCl₄): v=3580, 3066, 2951, 2922, 2850, 1724, 1425, 1384, 1365, 1152, 1107, 905; [α]_{D}²⁰=+70.7 (c=0.81, EtOAc); HRMS (ESI): m/z: calcd for C₄₁H₆₀NaO₄Si: 667.4159 [M+Na]⁺; found: 667.4128.

### EXAMPLE 37

2,2-Dimethyl-propionic acid [(4R,4aR,6R,7R,11R,12aR)-11-(tert-butyl-diphenyl-silanoxy)-7-hydroxy-4-isopropyl-1-methyl-3,4,4a,5,6,7,10,11,12,12a-decahydro-benzocyclodecen-6-yl]ester.

The compound prepared in Example 36 (18 mg, 0.028 mmol) was dissolved in degassed CH₂Cl₂ (2.3 mL). Grubbs-II (1 mg, 1.4 μmol) in degassed CH₂Cl₂ (0.5 mL) was slowly added. The reaction mixture stirred for 2 d at room temperature. Additional Grubbs-II (1 mg, 1.4 μmol) in degassed CH₂Cl₂ (0.2 mL) was slowly added and the mixture stirred for further 3 d at room temperature. Additional Grubbs-II (1 mg, 1.3 μmol) in degassed CH₂Cl₂ (0.2 mL) was slowly added and the mixture stirred for further 1 d at room temperature. The solvent was evaporated under reduced pressure and the residue was purified by flash chromatography (hexane/EtOAc 9:1) to provide unreacted starting compound (4 mg) and the title compound (10 mg, 55%, 66% after recovered starting material) as a colourless oils. R_{f}=0.20 (hexane/EtOAc 9:1); ¹H NMR (400 MHz, CDCl₃): δ=7.66-7.63 (m, 4H), 7.447.32 (m, 6H), 6.10-5.91 (m, 2H), 5.75-5.70 (m, 1H), 5.55-5.49 (m, 1H), 5.14-5.05 (m, 2H), 4.40-4.36 (m, 1H), 4.30-3.80 (m, 4H), 2.81-2.73 (m, 1H), 2.38-2.25 (m, 2H), 2.15-2.10 (m, 1H), 1.82-1.10 (m, 16H), 1.06 (s, 9H), 0.85 (d, J=6.8 Hz, 3H), 0.67 (d, J=6.8 Hz, 3H); ¹³C NMR (100.8 MHz, CDCl₃): δ=173.9, 136.3, 135.9 (2C), 135.8 (2C), 133.9 (2C), 130.3, 129.8, 129.7, 127.7, 127.6 (2C), 127.5 (2C), 119.3, 75.3, 73.1, 72.3, 37.2, 36.5, 35.1, 34.7, 30.5, 27.7, 27.2 (3C), 27.0 (3C), 26.4, 24.4, 22.7, 21.0 (2C), 20.8, 19.2; IR (CCl₄): v=3618, 3070, 2955, 2923, 2850, 1725, 1477, 1459, 1425, 1384, 1365, 1155, 1100, 1080; [α]_{D}²⁰=+4.0 (c=0.35, EtOAc); HRMS (ESI): m/z: calcd for C₃₉H₅₆NaO₄Si: 639.3846 (M+Na]⁺; found: 639.3860.

### EXAMPLE 38

Operating as Described in the Previous Examples, the Following Compounds are Prepared: AA 3-Phenyl-acrylic acid 11-acetoxy-1-isopropyl-4-methyl-1,2,4a,5,6,7,10,11,12,12a-decahydro-benzocyclodecen-6-yl ester. 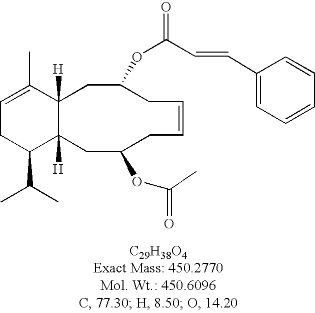
AB 3-(2-Methyl-thiazol-4-yl)-acrylic acid 11-acetoxy-1-isopropyl-4-methyl-1,2,4a,5,6,7,10,11,12,12a-decahydro-benzocyclodecen-6-yl ester. 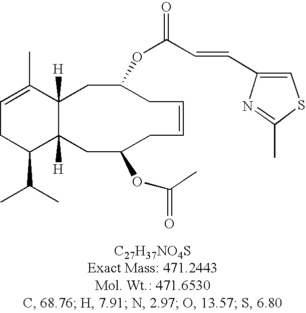
AC 3-(2-Methyl-oxazol-4-yl)-acrylic acid 11-acetoxy-1-isopropyl-4-methyl-1,2,4a,5,6,7,10,11,12,12a-decahydro-benzocyclodecen-6-yl ester. 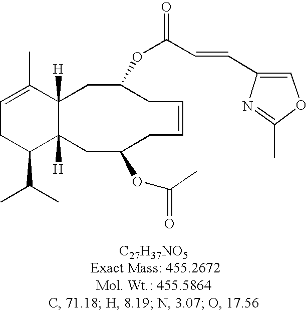
AD 3-Pyridin-2-yl-acrylic acid 11-acetoxy-1-isopropyl-4-methyl-1,2,4a,5,6,7,10,11,12,12a-decahydro-benzocyclodecen-6-yl ester. 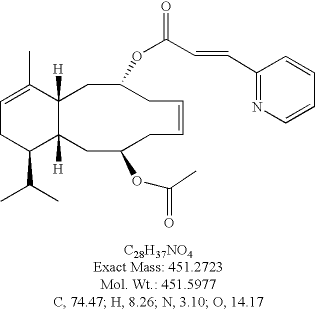
AE 3-(1-Methyl-1H-imidazol-4-yl)-acrylic acid 11-acetoxy-1-isopropyl-4-methyl-1,2,4a,5,6,7,10,11,12,12a-decahydro-benzocyclodecen-6-yl ester. 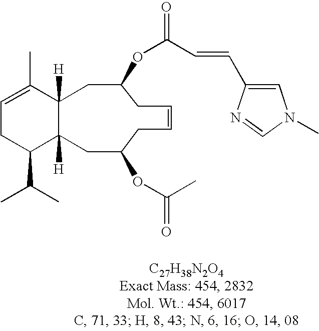
AF 3-(1-Methyl-1H-imidazol-4-yl)-acrylic acid 11-acetoxy-7-hydroxy-1-isopropyl-4-methyl-1,2,4a,5,6,7,10,11,12,12a-decahydro-benzocyclodecen-6-yl ester. 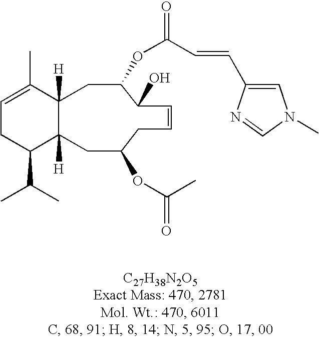
AG 3-(1-Methyl-1H-imidazol-4-yl)-acrylic acid 11-acetoxy-1-isopropyl-4-methyl-7-oxo-1,2,4a,5,6,7,10,11,12,12a-decahydro-benzocyclodecen-6-yl ester. 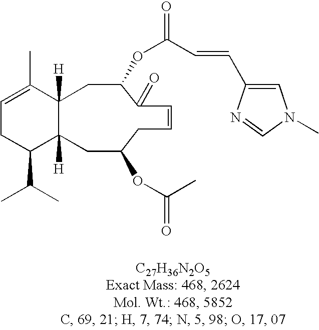
AH 3-(1-Methyl-1H-imidazol-4-yl)-acrylic acid 7,11-diacetoxy-1-isopropyl-4-methyl-1,2,4a,5,6,7,10,11,12,12a-decahydro-benzocyclodecen-6-yl ester. 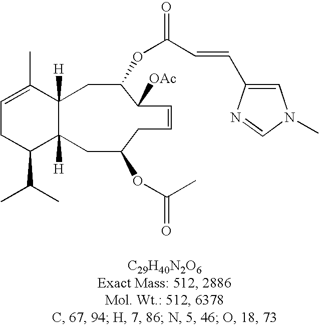
AI 3-(1-Methyl-1H-imidazol-1-yl)-acrylic acid 11-acetoxy-1-isopropyl-4-methyl-1,2,4,a5,6,7,8,9,10,11,12,12a-dodecahydro-benzocyclodecen-6-yl ester. 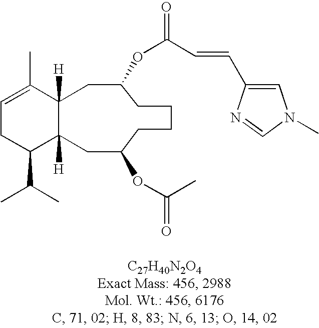
AL 3-(1-Methyl-1H-imidazol-4-yl)-acrylic acid 9-acetoxy-12-isopropyl-15-methyl-tricyclo[9,4,0,0^{5.7}]pentadec-14-en-3-yl ester. 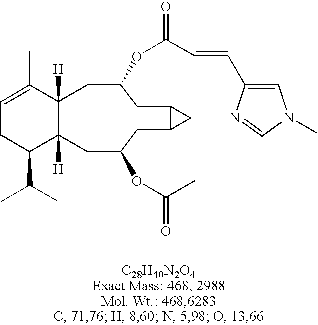
AM 3-(1-Methyl-1H-imidazol-4yl)-acrylic acid 11-acetoxy-10-hydroxy-1-isopropyl-4-methyl-1,2,4a,5,6,7,10,11,12,12a-decahydro-benzocyclodecen-6-yl ester. 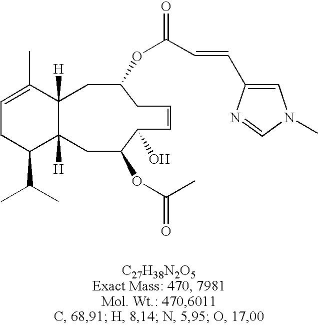
AN 3-(1-Methyl-1H-imidazol-4-yl)-acrylic acid 10,11-diacetoxy-1-isopropyl-4-methyl-1,2,4a,5,6,7,10,11,12,12a-decahydro-benzocyclodecen-6-yl ester. 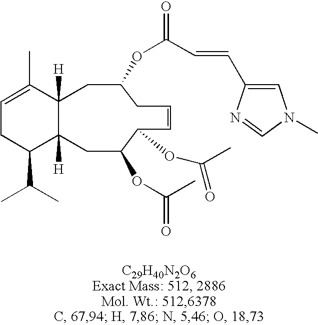
AO 3-(1-Methyl-1H-imidazol-4-yl)-acrylic acid 11-acetoxy-1-isopropyl-4methyl-10-oxo-1,2,4a,5,6,7,10,11,12,12a-decahydro-benzocyclodecen-6-yl ester. 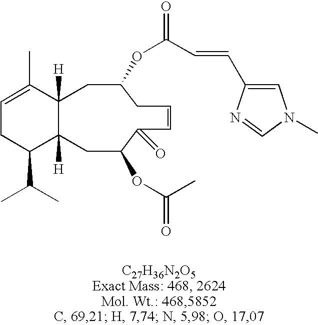
AP 3-(1-Methyl-1H-imidazol-4-yl)-acrylic acid 11-acetoxy-1-isopropyl-4-methyl-10-oxo-1,2,4a5,6,7,10,11,12,12a-decahydro-benzocyclodecen-6-yl ester. 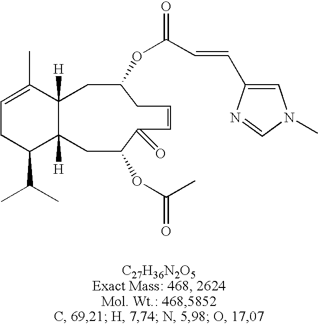
AQ 3-(1-Methyl-1H-imidazol-4-yl)-acrylic acid 11-acetoxy-10-hydroxy-1-isopropyl-4-methyl-1,2,4a,5,6,7,10,11,12,12a-decahydro-benzocyclodecen-6-yl ester 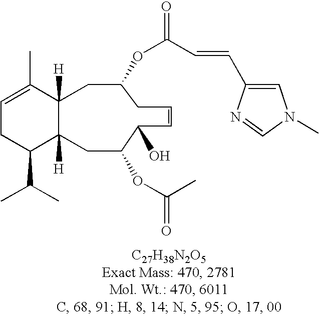
AR 3-(1-Methyl-1H-imidazol-4-yl)-acrylic acid 10,11-diacetoxy-1-isopropyl-4-methyl-1,2,4a,5,6,7,10,11,12,12a-decahydro-benzocyclodecen-6-yl ester. 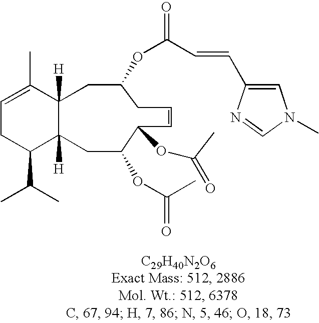
AS 3-(1-Methyl-1H-imidazol-4-yl)-acrylic acid 11-hydroxy-1-isopropyl)-4-methyl-1,2,4a,5,6,7,10,11,12,12a-decahydro-benzocyclodecen-6-yl ester. 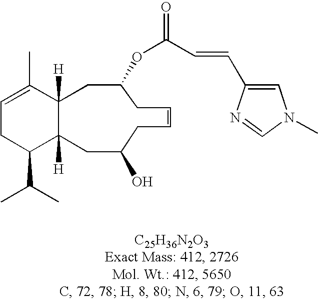
AT 3-(1-Methyl-1H-imidazol-4-yl)-acrylic acid 11-acetoxy-1-isopropyl-4-methyl-1,2,4a,5,6,7,10,11,12,12a-decahydro-benzocyclodecen-6-yl ester 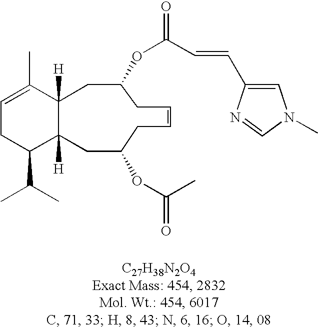
AU 3-(1-Methyl-1H-imidazol-4-yl)-acrylic acid 11-hydroxy-1-isopropyl-4-methyl-1,2,4a,5,6,7,10,11,12,12a-decahydro-benzocyclodecen-6-yl ester. 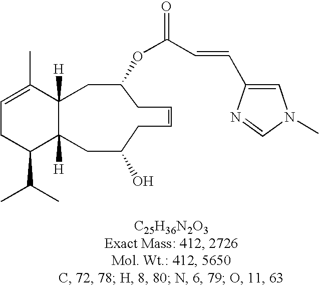
AV (E)-3-(1-Methyl-1H-imidazol-4-yl)-acrylic acid [(1R,4aR,6R,10R,11R,12aR)-11-(2,2-dimethyl-propionyloxy)-1-isopropyl-10-methoxy-4-methyl-1,2,4a,5,6,7,10,11,12,12a-decahydro-benzocyclodecen-6-yl]ester. 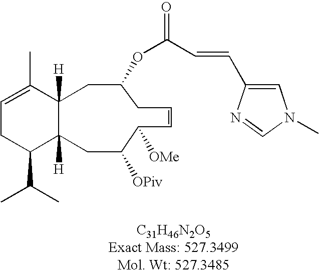
AX (E)-3-(1-Methyl-1H-imidazol-4-yl)-acrylic acid [(1R,4aR,6R,10R,11R,12aR)-11-(2.2-dimethyl-propionyloxy)-1-isopropyl-10-hydroxy-4-methyl-1,2,4a,5,6,7,10,11,12,12a-decahydro-benzocyclodecen-6-yl]ester. 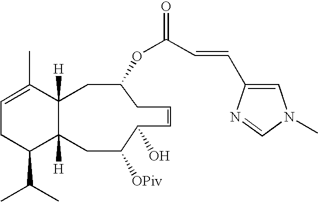
AY 3-(1-Methyl-1H-imidazol-4-yl)-acrylic acid 1-isopropyl-11-methoxycarbonylmethylene-4-methyl-1,2,4a,5,6,7,10,11,12,12a-decahydro-benzocyclodecen-6-yl ester. 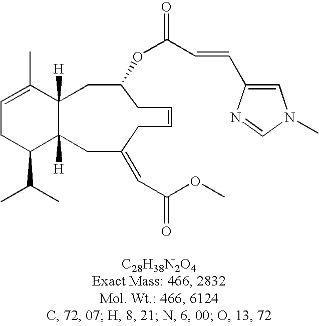
AW 3-(1-Methyl-1H-imidazol-4-yl)-acrylic acid 1-isopropyl-11-methoxymethylene-4-methyl-1,2,4a,5,6,7,10,11,12,12a-decahydro-benzocyclodecen-6-yl ester. 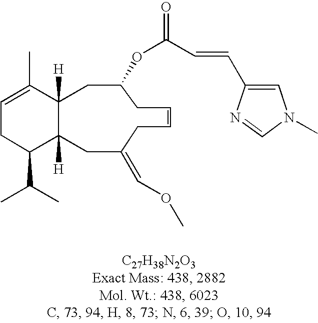
AZ 3-(1-Methyl-1H-imidazol-4-yl)-acrylic acid 11-ethoxycarbonylmethylene-1-isopropyl-4-methyl-1,2,4a,5,6,7,10,11,12,12a-decahydro-benzocyclodecen-6-yl ester. 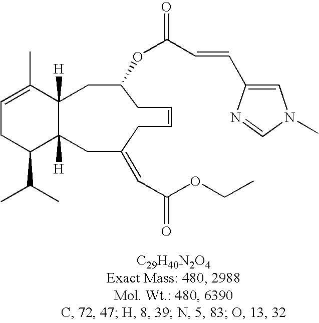
BA 3-(1-Methyl-1H-imidazol-4-yl)-acrglic acid 11-formyl-1-isopropyl-4-methyl-1,2,4a,5,6,7,10,11,12,12a-decahydro-benzocyclodecen-6-yl ester. 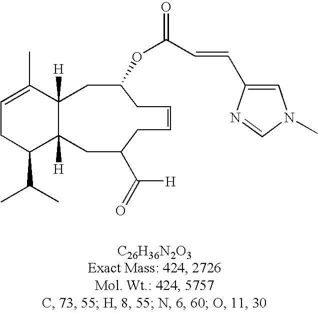
BB 3-(1-Methyl-1H-imidazol-4-yl)-acrylic acid 11-hydroxymethyl-1-isopropyl-4-methyl-1,2,4a,5,6,7,10,11,12,12a-decahydro-benzocyclodecen-6-yl ester. 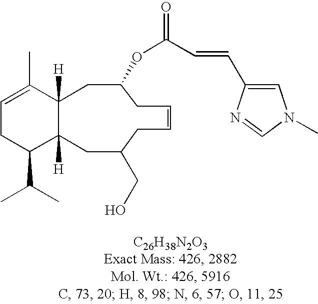
BC 3-(1-Methyl-1H-imidazol-4-yl)-acrylic acid 11-(3-acetoxy-4,5-dihydroxy-tetrahydro-pyran-2-yloxymethyl)-1-isopropyl-4-methyl-1,2,4a,5,6,7,10,11,12,12a-decahydro-benzocyclodecen-6-yl ester. 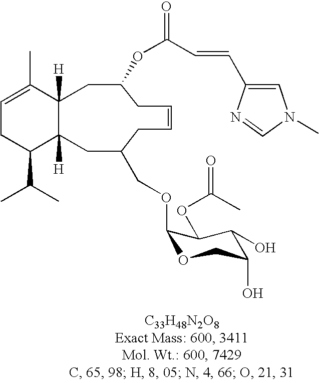
BD 4-Isopropyl-1-methyl-11-[3-(1-methyl-1H-imidazol-4-yl)-acryloyloxy]-3,4,4a,5,6,7,10,11,12,12a-decahydro-benzocyclodecene-6-carboxylic acid ethyl ester. 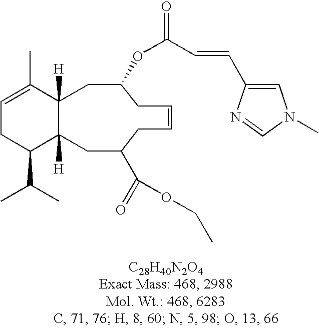
BE 7-Hydroxy-4-isopropyl-1-methyl-11-[3-(1-methyl-1H-imidazol-4-yl)-acryloyloxy]-3,4,4a,7,10,11,12,12a-octahydro-benzocyclodecene-6-carboxylic acid ethyl ester. 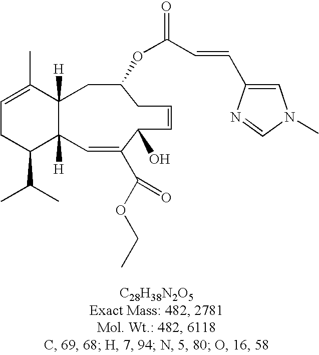
BF 7-Hydroxy-4-isopropyl-1-methyl-11-[3-(1-methyl-1H-imidazol-4-yl)-acryloyloxy]-3,4,4a,7,10,11,12,12a-octahydro-benzocyclodecene-6-carboxylic acid ethyl ester. 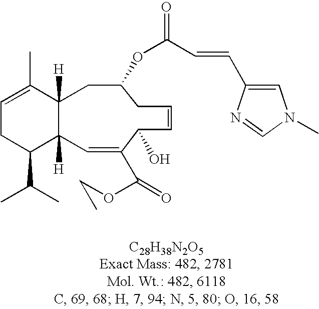
BG (E)-3-(1-Methyl-1H-imidazol-4-yl)-acrylic acid [(1R,4a,6R,11R,12aR)-11-(2,2-dimethyl-propionyloxy)-1-isopropyl-10-oxo-4-methyl-1,2,4a,5,6,7,10,11,12,12a-decahydro-benzocyclodecen-6-yl]ester. 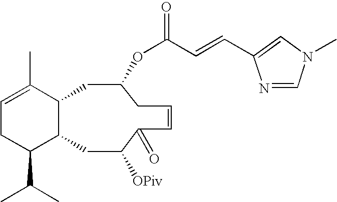
BH (E)-3-(1-Methyl-1H-imidazol-4-yl)-acrylic acid [(1R,4aR,6R,10R,11R,12aR)-11-(2.2-dimethyl-propionyloxy)-1-isopropyl-10-acetoxy-4-methyl-1,2,4a,5,6,7,10,11,12,12a-decahydro-benzocyclodecen-6-yl]ester.

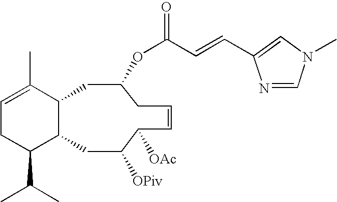

1. A compound which is a sarcodictyin derivative having formula I: 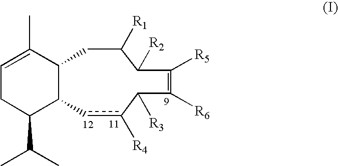
wherein: 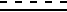
at positions 8-9 and 11-12 independently represents a single or double bond, —R₁ represents oxygen (═O), or a residue —OR₇, wherein R₇ represents hydrogen, linear or branched C₁-C₇ alkanoyl, benzoyl, C₁-C₁₀ alkyl, C₂-C₁₀ alkenyl or a residue of the formula 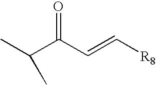
wherein R₈ is an optionally substituted aryl or heterocyclyl;
one of —R₂ and —R₃ represents hydrogen and the other one is chosen from the group consisting of hydrogen, oxygen (═O) and a residue —OR₉, wherein R₉ represents hydrogen, C₁-C₆ alkanoyl or benzoyl;
when 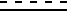
at position 11-12 represents a single bond, then —R₄ represents oxygen (═O), methylene (═CH2), =CHCOOR₁₀, wherein R₁₀ represents C₁-C₁₀ alkyl or optionally substituted aryl; ═CH(OCH₃), or a residue of formula
—OR₉, wherein R₉ is as defined above;
—CH₂OR₁₁ wherein R₁₁ represents hydrogen or a sugar residue, or
—COR₁₂ wherein R₁₂ represents hydrogen, —OH or —OR₁₀, wherein R₁₀ is as defined above; or
when 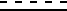
at position 11-12 represents a double bond, then —R₄ represents a residue of formula —CH₂OR₁₁ or —COR₁₂ as defined above; —R₅ and —R₆ are both hydrogen or, when 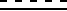
at position 8-9 represents a single bond, taken together with the carbon atoms to which they are attached form a cyclopropane ring; or a pharmaceutically acceptable salt thereof.

2. A compound according to claim 1 wherein the sarcodictyin derivative is of formula Ia 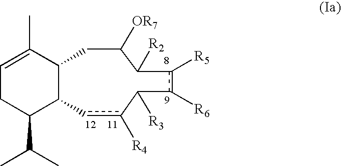
(Ia) wherein: 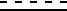
at positions 8-9 and 11-12 independently represents a single or double bond, R₇ represents a residue of the formula 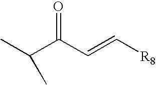
wherein R₈ is N-methyl imidazolyl, phenyl, methyl-thiazolyl, methyl-oxazolyl or pyridyl group;
one of —R₂ and —R₃ represents hydrogen and the other one is hydrogen or oxygen (═O), hydroxy or acetoxy;
when 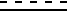
at position 11-12 represents a single bond, then —R₄ represents oxygen (═O), methylene (═CH₂), ═CHCOOR₁₀, wherein R₁₀ represents methyl or ethyl, ═CH(OCH₃), —CHO;
hydroxy, acetoxy, pivaloyloxy or —CH₂OR₁₁ wherein R₁₁ represents hydrogen or a sugar residue having the formula 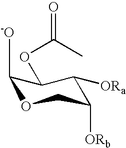
wherein Rₐ and R_{b} independently represent hydrogen, a hydroxy protecting group, or C₁-C₆ alkanoyl, or, when 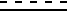
at position 11-12 represents a double bond, then —R₄ represents a residue of formula —CO₂C₂H₅; and
R₅ and —R₆ are both hydrogen or, when 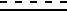
at position 8-9 represents a single bond, taken together with the carbon atoms to which they are attached form a cyclopropane ring.

3. A compound as claimed in claim 2 wherein the substituent OR₇ in formula Ia is under the plane and the substituent R₄ is above the plane; R₂ and R₃ are hydrogen and RB is N-methyl imidazolyl.

4. A process for producing a compound as defined in claim 1, which process comprises cyclizing a compound of formula II 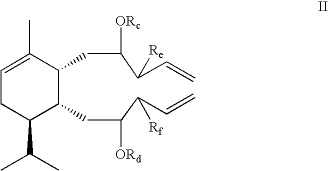
wherein R, represents hydrogen, a silyl protecting group, C₁-C₆ alkanoyl or benzoyl or, taken together with Rₑ, forms an acetonide ring; R_{d} represents hydrogen, C₁-C₆ alkanoyl, or benzoyl, or, taken together with R_{f}, forms an acetonide ring; either Rₑ represents H and R_{f} either represents OH or is linked to the adjacent OR_{d} substituent as defined above, or R_{f} represents H and Rₑ either represents OH or is linked to the adjacent OR_{c} substituent as defined above;
and, if desired, converting one resulting sarcodictyin derivative of formula I′, 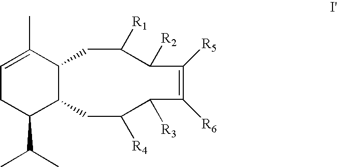
wherein R₁ is OR_{c}, R₂ is Rₑ, R₃ is R_{f}, R₄ is OR_{d}, in which R_{c}, R_{d}, Rₑ and R_{f} are as defined above and R₅ and R₆ are hydrogen, into another sarcodictyin derivative of formula I as defined in claim 1 and/or, if desired, converting a sarcodictyin derivative of formula I′ or I into a pharmaceutically acceptable salt thereof; and/or, if desired converting a pharmaceutically acceptable salt of a sarcodictyin derivative of formula I or I′ into the corresponding free compound.

5. A process according to claim 4 wherein the cyclization is carried out through the Ring Closing Metathesis (RCM) reaction.

6. A process according to claim 5 in which the RCM reaction is carried out in the presence of a Nolan and Grubb's catalyst

7. A pharmaceutical composition which comprises a pharmaceutically acceptable diluent or carrier and, as an active ingredient, a compound as defined in any; of claims claim 1.

8. (Canceled)

9. (Canceled)

10. (Canceled)

11. A method of treating a patient in need of an antitumour agent, which method comprises the administration thereto of a compound as defined claim 1.

12. A method according to claim 11 wherein the patient is suffering from leukemia or a solid tumour.

13. A pharmaceutical composition which comprises a pharmaceutically acceptable diluent or carrier and, as an active ingredient, a compound as defined in claim 2.

14. A pharmaceutical composition which comprises a pharmaceutically acceptable diluent or carrier and, as an active ingredient, a compound as defined in claim 3.

15. A method of treating a patient in need of an antitumour agent, which method comprises the administration thereto of a compound as defined in claim 2.

16. A method according to claim 15 wherein the patient is suffering from leukemia or a solid tumour.

17. A method of treating a patient in need of an antitumour agent, which method comprises the administration thereto of a compound as defined in claim 3.

18. A method according to claim 17 wherein the patient is suffering from leukemia or a solid tumour.
